(19) United States
(12) Patent Application Publication
(10) Pub. No.: US 2018/0192939 A1
(43) Pub. Date: Jul. 12, 2018

(54) MEDICAL DEVICES WITH INTEGRATED SENSORS AND METHOD OF PRODUCTION

(71) Applicant: MiRus LLC, Atlanta, GA (US)

(72) Inventors: Noah Roth, Atlanta, GA (US); Jay Yadav, Sandy Springs, GA (US)

(21) Appl. No.: 15/741,347

(22) PCT Filed: Jul. 1, 2016
(86) PCT No.: PCT/US2016/040633

§ 371 Date: Jan. 2, 2018

Related U.S. Application Data
(60) Provisional application No. 62/187,863, filed on Jul. 2, 2015.
(60) Provisional application No. 62/304,092, filed on Mar. 4, 2016.
(60) Provisional application No. 62/304,088, filed on Mar. 4, 2016.

(51) Int. Cl. A61B 5/00; A61B 17/70; A61F 2/44

(52) U.S. Cl. CPC A61B 5/4561; A61B 17/7002; A61B 2017/00526; A61B 17/7065; A61F 2/4455; A61B 17/7064

(57) ABSTRACT

Medical devices used for the treatment of disease, monitoring of physiological properties and the correction of deformities and/or degenerative conditions including integrated sensors providing physiological parameters to assist in assessing healing and the clinical management of patients. Including methods of production of said medical devices.

### CROSS-REFERENCE TO RELATED APPLICATIONS

This application claims the benefit of U.S. provisional patent application No. 62/187,863, filed on Jul. 2, 2015, and entitled “MEDICAL DEVICES WITH INTEGRATED SENSORS AND METHOD OF PRODUCTION;” U.S. provisional patent application No. 62/304,092, filed on Mar. 4, 2016, and entitled “VERTEBRAL BODY STENT;” and U.S. provisional patent application No. 62/304,088, filed on Mar. 4, 2016, and entitled “PEDICLE SCREW ROD HAVING A STRAIN SENSOR,” the disclosures of which are expressly incorporated herein by reference in their entireties.

### FIELD OF THE INVENTION

The present disclosure relates to medical devices in general and diagnostic tools used to provide physiological parameters for management of patients suffering from one or more medical conditions. More particularly, the present disclosure relates to the integration of sensors into such devices and the methods of use and production of such devices.

### BACKGROUND

Medical devices and diagnostic tools have long been used for the treatment of disease and monitoring of physiological parameters, respectively. In recent years medical devices have sought to integrate diagnostic measurements of physiological parameters to assist in the clinical management of disease and in determining the effectiveness of a particular treatment, whether by therapeutic agent and/or medical device.

However, the prior art devices have been limited to devices specific to cardiac conditions. Nonetheless, the methods of manufacture of integrated devices within the field of cardiology failed to make use of integrated manufacturing procedures relying on traditional multi-step manufacturing processes, thus being labor intensive and requiring a high level of skill by the operator.

### SUMMARY

Advancements in rapid production of devices and the ability to construct a single device with one or more materials, surface characteristics, and the integration of an individual component within a secondary device has provided the opportunity to create a truly integrated system. One such non-limiting example is the use of 3D printing technology whereby an apparatus can be constructed in a cost effective manner using plastics, metals, ceramics, or combinations thereof to construct integrated and highly complex devices in a one-step operation.

One non-limiting example of a medical device of the present disclosure that can be used to treat a disease is a device such as a spinal cage used in the treatment of lordosis, characterized by an increase in the inward curvature of the spine. The spinal cage is used to correct the curvature of the spine and/or fuse the lower and upper vertebral bodies at the correct curvature thereby providing a normal curvature of the spine. The spinal cages can optionally be implanted with bone morphogenetic proteins (BMP) to assist in fusion of the vertebral bodies. Although BMP is used to facilitate in fusion, the rate of fusion is highly dependent upon individual patients and present comorbidities. Integration of sensors (e.g., in the form of a strain gauge, etc.) into the spinal cage could be used to provide the clinician with a real-time and accurate method of determining when fusion has occurred (i.e., when no further fluctuations of the strain gauge are detected, etc.). Furthermore, the use of manufacturing means to make the spinal cage with the sensor integrated within the device without compromising the function and effectiveness of the spinal cage is also a feature of the present disclosure.

A method for treating a condition of the spine is described herein. The method can be used to treat abnormal curvature of the spine, for example. The method can include inserting a stent within a space between a two vertebral bodies, and expanding the stent to move at least one of the vertebral bodies. The expanded stent can reset a listing height between the vertebral bodies and curvature of the vertebral bodies.

In some implementations, the curvature is lordotic curvature. Alternatively, in other implementations, the curvature can be kyphotic curvature.

Alternatively or additionally, the stent can be a catheter mounted stent.

Alternatively or additionally, the stent can have a mesh pattern.

An example pedicle screw rod is described herein. The pedicle screw rod can include an elongate rod, and a sensor configured to measure fusion of one or more vertebral bodies as a function of strain in the elongate rod.

In some implementations, the sensor can be attached to or on a portion of the elongate rod. Alternatively or additionally, the sensor can be integrated with a surface of the elongate rod.

Alternatively or additionally, the sensor can be a strain gauge. Alternatively or additionally, the sensor can include a piezoelectric or piezoresistive element.

Alternatively or additionally, the pedicle screw rod can include a plurality of sensors.

An example intervertebral spacer is described herein. The intervertebral spacer can include a structure configured for insertion between vertebral bodies to reset listing height, and a sensor configured to measure fusion of one or more vertebral bodies as a function of strain.

Alternatively or additionally, the sensor can optionally be mounted on upper or lower face of the intervertebral spacer. Alternatively or additionally, the sensor can optionally be integrated within the intervertebral spacer. Alternatively or additionally, the sensor can optionally be printed on the upper or lower face of the intervertebral spacer or between the upper and lower face of the intervertebral spacer.

Alternatively or additionally, the sensor can be a strain gauge. Alternatively or additionally, the sensor can include a piezoelectric or piezoresistive element.

Alternatively or additionally, the intervertebral spacer can include a plurality of sensors.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

Exemplary embodiments of the present disclosure that are shown in the figures are summarized below. These and other embodiments are more fully described in the Detailed Description section. It is to be understood, however, that there is no intention to limit the disclosure to the forms described within this application. One skilled in the art can recognize that there are numerous modifications, equivalents and alter-native constructions that fall within the spirit and scope of the disclosure.

FIG. 1, FIG. 2, FIG. 3, and FIG. 4 illustrate the primary clinical modalities resulting in a loss of angulation 600 and gap 610 between the vertebral bodies 620 in relation to each other as shown in FIG. 6. FIG. 1 illustrates a normal spine (left side) and lordosis of the spine (right side). FIG. 2 illustrates a normal spine (left side) and a kyphotic spine (right side). FIG. 3 illustrates normal cartilage 300, degenerated cartilage 310, and a collapsed disc 320. FIG. 4 illustrates a herniated disc 400 and compressed lumbar spinal nerve 410. FIG. 5 illustrates one non-limiting embodiment of a spinal cage (e.g., an intervertebral spacer) used to correct the loss of angulation.

FIG. 7 depicts a spinal cage (e.g., an intervertebral spacer) with a sensor associated with the spinal cage in one non-limiting surface of the spinal cage and the use of a wired or wireless connection to the sensor to detect changes in pressure and transmit such detected information externally for use in evaluating the fixation, stabilization or correction or combination thereof of the clinical modality as described within this application.

FIG. 8 depicts a spinal cage (e.g., an intervertebral spacer) with a sensor associated with the spinal cage in one non-limiting cross-section (i.e., an imbedded gauge) of the spinal cage, and the use of a wired or wireless connection to the sensor to detect changes in pressure and transmit such detected information externally for use in evaluating the fixation, stabilization or correction or combination thereof of the clinical modality as described within this application.

FIG. 9 depicts one non-limiting graphical display 900 of the relative strain on the gauge via a surface or imbedded sensor from which to evaluate fixation, stabilization or correction or combinations thereof of the clinical modality. As in one non-limiting example, the shading shown within FIG. 9 would be interpreted to show the highest pressure/strain on the front (anterior) portion of the spinal cage and the lowest pressure/strain on the back (posterior) portion of the spinal cage. The shading scheme shown would indicate a lack of uniformity in the pressure/strain of the sensor which would translate into a variation in the contact of the spinal cage with the vertebral plates of the corresponding vertebral bodies and providing clinical input from which to correct fixation and evaluate post-surgical stabilization and/or correction.

FIG. 10 illustrates an example where a condition of the spine is treated using a vertebral body stent.

FIG. 11 illustrates an example pedicle screw rod.

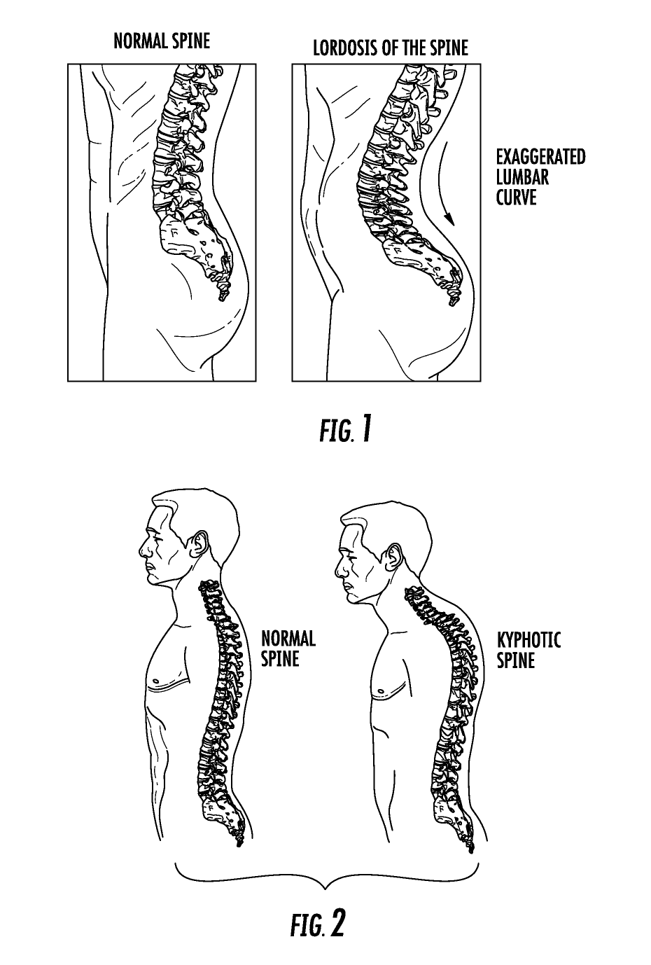

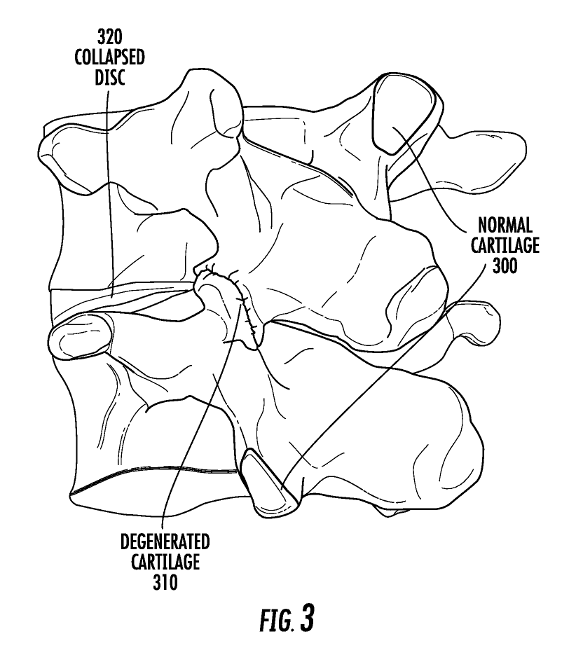

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

For purposes of the present disclosure, the term therapeutic agent is defined as any pharmaceutical, biologic, or substance effecting irregularities in the normal physiology of a living organism, their derivatives, analogs, or combinations thereof, whether administered orally, topically, sub-dermally or by any other means used for the administration of therapeutic agents as defined herein and within normal nomenclature.

For purposes of the present disclosure, a medical device is any apparatus used in the treatment of diseases, deformity, degeneration, or combinations thereof, and/or any other medical condition regardless of being implanted or the duration of implantation.

Exemplary embodiments of the present disclosure that are shown in the figures are summarized below. These and other embodiments are more fully described below. It is to be understood, however, that there is no intention to limit the disclosure to the forms described within this application. One skilled in the art can recognize that there are numerous modifications, equivalents and alter-native constructions that fall within the spirit and scope of the disclosure.

For purposes of the present disclosure, a spinal cage will be used to for descriptive purposes only and is not meant to limit the scope of this disclosure nor its application to devices in non-orthopedic applications.

Spinal cages are devices implanted between the vertebral bodies of the spine for purposes of correcting the curvature of the spine and the associated lordosis (excessive inward curvature of the spine), kyphosis (excessive outward curvature of the spine), disk herniation, disk collapse or combination thereof. Spinal cages are supplied in various different angles to not only correct any deficiencies in the height between the vertebral bodies, but to also correct the angulation of the vertebral plate with respect to each other.

The stages of implantation and correction can be characterized by: 1) fixation of the spinal cage between the vertebral bodies having adequate contact between the vertebral plates and the surface of the spinal cage (Note: fixation is also associated with the implantation of BMP); 2) stabilization of the vertebral bodies with respect to each other, which is associated with fixation of the spinal cage and the implantation of bone cement to bind the spinal cage between the vertebral bodies and associated with the ability to provide normal loading to the spine (e.g., minor physical activity); and 3) correction which is characterized as fusion of the two vertebral bodies and the resumption of normal activities. The spinal cages are supplied in multiple sizes and shapes and may contact anywhere from 25% to 75% of the surface area of the vertebral plates of the vertebral bodies intended to be joined.

Each stage of implantation and correction is associated with its own risks and the clinician uses experience and/or anecdotal evidence of the spinal cage and its performance to determine when each stage of the correction has been properly completed. For example, it is well known that the angulation of the spinal cage does not always match the natural angulation of the spine and one or more portions of the spinal cage may not make contact with the vertebral plates, thus potentially leading to a complication requiring an increased hospital stay or revision surgery. Although surgeons have some level of visualization during implantation and the use of fluoroscopy to visualize placement of the spinal cage, both these methods lack quantitative evidence of accurate fixation and have limitations associated with stabilization and correction.

A spinal cage with one or more sensors has the potential to provide accurate confirmation of fixation between the vertebral bodies, as well as to provide continuous data for an accurate determination of stabilization and/or correction.

In one non-limiting example, one or more of the integrated sensor(s) can be placed on one or more surfaces of the medical device (e.g., spinal cage, etc.) and/or within the cross-section or interior of the medical device. The sensor can provide quantitative data from the medical device (e.g., providing strain values correlated to contact with the vertebral bodies, stabilization of the implanted cage and vertebral body construct and correction (i.e., fusion), etc.). In an alternate non-limiting embodiment, the strain values can be used to create a three dimensional image showing contact points between the medical device (e.g., spinal cage, etc.) and the vertebral plates at time of implantation and post implantation. Strain values can be transmitted to a display via a wired connection that can be designed to be removable and/or using a radio frequency or other wireless signal transmission.

The spinal cage as described herein in one embodiment of the disclosure is traditionally made using CNC machining techniques, and the sensor(s) generally need to be attached to the spinal cage post machining. Traditional production techniques require sectioning the spinal cage and implantation of a sensor(s) within the body of the spinal cage, and then re-sealing the spinal cage, if there was a need or desire to have the gauge integrated within the body of the spinal cage.

Rapid production techniques have been developed to prototype medical devices prior to investing in dedicated production machinery. However, in recent years, these prototype techniques have been increasingly viewed as production processes for the finished device. More specifically, 3D printing has gone far beyond the prototype stage and is able to produced finished devices out of a host of materials (e.g., metals, plastics, composite materials, etc.), and a single 3D printer can be set up to print one or more plastics and one or more metals and use various materials to create a finished part. Metal parts traditionally require thermo sintering to eliminate voids in the material, which creates an issue when used in conjunction with plastics. For example, the sintering temperature of metals is beyond the melting or transition temperature of most plastics, causing irreparable harm to the finished device. However, alternate sintering technologies (light, laser, electrical, etc.) have been developed allowing for 3D printing metal within or as part of a plastic part to be sintering, thereby increasing the final product's mechanical integrity and ability to conduct electricity.

Alternately, there are a new group of conductive polymers providing stretchable and mechanically robust electrical circuits in conjunction with a polymeric or metallic part. Poly(3,4-ethylenedioxythiophene):poly(4-styrenesulfonate) or PEDOT:PSS is one such polymer. Conductive polymers like polyanilines, polypyrrols and polythiophenes become conductive by removing an electron from their conjugated n-orbitals via doping. The electrical conductivity results from the delocalization of electrons along the polymer backbone; hence, the term “synthetic metals”. Clevios™ PEDOT:PSS is another substituted polythiophene ionomer complex with a polyanion that offers the highest conductivity found so far in a commercial product. The product is offered as the monomer for in-situ polymerization, as neat water-based dispersions or as ready-to-use formulations mixed with solvents and additives.

The medical device can optionally be at least partially made of an alloy having improved properties as compared to past medical devices. The alloy used to at least partially form the medical device improves one or more properties (e.g., strength, durability, hardness, biostability, bendability, coefficient of friction, radial strength, flexibility, tensile strength, tensile elongation, longitudinal lengthening, stress-strain properties, improved recoil properties, radiopacity, heat sensitivity, biocompatibility, improved fatigue life, crack resistance, crack propagation resistance, etc.) of such medical device. These one or more improved physical properties of the alloy can be achieved in the medical device without having to increase the bulk, volume and/or weight of the medical device, and in some instances these improved physical properties can be obtained even when the volume, bulk and/or weight of the medical device is reduced as compared to medical devices that are at least partially formed from traditional stainless steel materials. However, it will be appreciated that the alloy can include metals such as stainless steel, etc.

The alloy that is used to at least partially form the medical device can thus 1) increase the radiopacity of the medical device, 2) increase the radial strength of the medical device, 3) increase the yield strength and/or ultimate tensile strength of the medical device, 4) improve the stress-strain properties of the medical device, 5) improve the crimping and/or expansion properties of the medical device, 6) improve the bendability and/or flexibility of the medical device, 7) improve the strength and/or durability of the medical device, 8) increase the hardness of the medical device, 9) improve the longitudinal lengthening properties of the medical device, 10) improve the recoil properties of the medical device, 11) improve the friction coefficient of the medical device, 12) improve the heat sensitivity properties of the medical device, 13) improve the biostability and/or biocompatibility properties of the medical device, 14) increase fatigue resistance of the medical device, 15) resist cracking in the medical device and resist propagation of crack, and/or 16) enable smaller, thinner and/or lighter weight medical devices to be made. The medical device generally includes one or more materials that impart the desired properties to the medical device so as to withstand the manufacturing processes that are needed to produce the medical device. These manufacturing processes can include, but are not limited to, laser cutting, etching, crimping, annealing, drawing, pickling, electroplating, electro-polishing, chemical polishing, cleaning, pickling, ion beam deposition or implantation, sputter coating, vacuum deposition, etc.

In another non-limiting aspect of the present disclosure, a medical device that can include the alloy is an orthopedic device, PFO (patent foramen ovale) device, stent, valve, spinal implant, vascular implant; graft, guide wire, sheath, stent catheter, electrophysiology catheter, hypotube, catheter, staple, cutting device, any type of implant, pacemaker, dental implant, bone implant, prosthetic implant or device to repair, replace and/or support a bone (e.g., acromion, atlas, axis, calcaneus, carpus, clavicle, coccyx, epicondyle, epitrochlea, femur, fibula, frontal bone, greater trochanter, humerus, ilium, ischium, mandible, maxilla, metacarpus, metatarsus, occipital bone, olecranon, parietal bone, patella, phalanx, radius, ribs, sacrum, scapula, sternum, talus, tarsus, temporal bone, tibia, ulna, zygomatic bone, etc.) and/or cartilage, nail, rod, screw, post, cage, plate, pedicle screw, cap, hinge, joint system, wire, anchor, spacer, shaft, spinal implant, anchor, disk, ball, tension band, locking connector, or other structural assembly that is used in a body to support a structure, mount a structure and/or repair a structure in a body such as, but not limited to, a human body. In one non-limiting application, the medical device is a dental implant dental filling, dental tooth cap, dental bridge, braces for teeth, dental teeth cleaning equipment, and/or any other medical device used in the dental or orthodontist field. In another non-limiting application, the medical device is a stent. In still another non-limiting application, the medical device is a spinal implant. In yet another non-limiting application, the medical device is a prosthetic device. Although the present disclosure will be described with particular reference to medical devices, it will be appreciated that the alloy can be used in other components that are subjected to stresses that can lead to cracking and fatigue failure (e.g., automotive parts, springs, aerospace parts, industrial machinery, etc.). The metals that are used to form the alloy are non-limiting. Generally, such metals include, nickel and chromium and one or more alloying agents such as, but are not limited to, aluminum, calcium, carbon, cerium oxide, cobalt, copper, gold, hafnium, iron, lanthanum oxide, lead, magnesium, molybdenum, niobium, osmium, platinum, rare earth metals, rhenium, silver, tantalum, technetium, titanium, tungsten, vanadium, yttrium, yttrium oxide, zinc, zirconium, zirconium oxide, and/or alloys of one or more of such components (e.g., MoHfC, MoY₂O₃, MoCs₂O, MoW, MoTa, MoZrO₂, MOLa₂O₃, MoRe alloy, etc.).

In still another non-limiting aspect of the present disclosure, carbon nanotubes (CNT) can optionally be incorporated into a metal material to form the alloy. Although the alloy is described as including one or more metals and/or metal oxides, it can be appreciated that some or all of the metal and/or metal oxide in the alloy can be substituted for one or more materials selected from the group of ceramics, plastics, thermoplastics, thermosets, rubbers, laminates, non-wovens, etc. The one or more metals used in the alloy generally have an alloy matrix and the CNT can be optionally incorporated within the grain structure of the alloy matrix. It is believed that certain portions of the CNT, when used, will cross the grain boundary of the metal material and embed into the neighboring grains, thus forming an additional linkage between the grains. When an alloy is employed in dynamic application, a cyclic stress is applied on the alloy. At some point at a number of cycles, the alloy will crack due to fatigue failure that initiates and propagates along the grain boundaries. It is believed that the attachment of CNT across the grains will prevent or prolong crack propagation and fatigue failure. Further, when the grain size is large, then the CNT gets completely embedded into a grain. The twinning of the grains is limited by the presence of CNT either fully embedded or partially embedded within the grain structure. Additionally, the CNT offers better surface erosion resistance. The alloy that includes the CNT can be made by powder metallurgy by adding the CNT to the metal powder or mixture of various metal powders to make a multicomponent alloy. The mixture can then be compressed under high isostatic pressure into a preform where the particles of the powder fuse together and thereby trap the CNT into the matrix of the alloy. The preform can then be sintered under inert atmosphere or reducing atmosphere and at temperatures that will allow the metallic components to fuse and solidify. Depending on the desired grain structure, the fused metal can then be annealed or further processed into the final shape and then annealed. At no point should the alloy be heated above 300° C. without enclosing the alloy in an inert or reducing atmosphere and/or under vacuum. The material can also be processed in several other conventional ways. One in particular will be by metal injection molding or metal molding technique in which the metal and CNT are mixed with a binder to form a slurry. The slurry is then injected under pressure into a mold of desired shape. The slurry sets in the mold and is then removed. The binder is then sintered off in multiple steps leaving behind the densified metal-CNT composite. The alloy may be heated up to 1500° C. in an inert or reducing atmosphere and/or under vacuum. Most elemental metals and alloys have a fatigue life which limits its use in a dynamic application where cyclic load is applied during its use. The alloy prolongs the fatigue life of the medical device. The alloy is believed to have enhanced fatigue life, enhancing the bond strength between grain boundaries of the metal in the alloy, thus, inhibiting, preventing or prolonging the initiation and propagation of cracking that leads to fatigue failure. For example, in an orthopedic spinal application, the spinal rod implant undergoes repeated cycles throughout the patient's life and can potentially cause the spinal rod to crack. Titanium is commonly used in such devices; however, titanium has low fatigue resistance. The fatigue resistance can be improved by alloying the titanium metal with CNT in the manner described above. If higher strength as well as higher fatigue resistance is required, then the CNT can be alloyed with alloy to obtain such properties. With the addition of at least about 0.05 weight percent, typically at least about 0.5 weight percent, and more typically about 0.5-5% weight percent of CNT to the metal material of the alloy, the alloy can exhibit enhanced fatigue life.

In another and/or alternative non-limiting aspect of the present disclosure, the medical device is generally designed to include at least about 25 weight percent of the metal alloy (i.e., 25%, 25.01%, 25.02% . . . 99.98%, 99.99%, 100% and any value or range therebetween); however, this is not required. In one non-limiting embodiment of the disclosure, the medical device includes at least about 40 weight percent of the metal alloy. In another and/or alternative non-limiting embodiment of the disclosure, the medical device includes at least about 50 weight percent of the metal alloy. In still another and/or alternative non-limiting embodiment of the disclosure, the medical device includes at least about 60 weight percent of the metal alloy. In yet another and/or alternative non- limiting embodiment of the disclosure, the medical device includes at least about 70 weight percent of the metal alloy. In still yet another and/or alternative non-limiting embodiment of the disclosure, the medical device includes at least about 85 weight percent of the metal alloy. In a further and/or alternative non-limiting embodiment of the disclosure, the medical device includes at least about 90 weight percent of the metal alloy. In still a further and/or alternative non-limiting embodiment of the disclosure, the medical device includes at least about 95 weight percent of the metal alloy. In yet a further and/or alternative non-limiting embodiment of the disclosure, the medical device includes about 100 weight percent of the metal alloy.

In still another and/or alternative non-limiting aspect of the present disclosure, the metal alloy that is used to form all or part of the medical device 1) is not clad, metal sprayed, plated and/or formed (e.g., cold worked, hot worked, etc.) onto another metal, or 2) does not have another metal or metal alloy metal sprayed, plated, clad and/or formed onto the metal alloy. It will be appreciated that in some applications, the metal alloy of the present disclosure may be clad, metal sprayed, plated and/or formed onto another metal, or another metal or metal alloy may be plated, metal sprayed, clad and/or formed onto the metal alloy when forming all or a portion of a medical device.

In yet another and/or alternative non-limiting aspect of the present disclosure, the alloy can be used to form a coating on a portion of all of a medical device. For example, the alloy can be used as a coating on articulation points of artificial joints. Such a coating can provide the benefit of better wear, scratch resistance, and/or elimination of leaching harmful metallic ions (i.e., Co, Cr, etc.) from the articulating surfaces when they undergo fretting (i.e., scratching during relative motion). As can be appreciated, the alloy can have other or additional advantages. As can also be appreciated, the alloy can be coated on other or additional types of medical devices. The coating thickness of the alloy is non-limiting. In one non-limiting example, there is provided a medical device in the form of a clad rod wherein in the core of the rod is formed of a metal or alloy (e.g., MoHfC, MoY₂O₃, MoCs₂O, MoW, MoTa, MoZrO₂, MoRe alloy, NiCoCrMo alloy, NiCrMoTi alloy, NiCrCuNb alloy, TiAlV alloy, etc.) or ceramic or composite material, and the other layer of the clad rod is formed of the alloy. The core and the other layer of the rod can each form 50-99% of the overall cross section of the rod. As can also be appreciated, the alloy can form the outer layer of other or additional types of medical devices. The coating can be used to create a hard surface on the medical device at specific locations as well as all over the surface. The base hardness of alloy can be as low as 300 Vickers and/or as high as 500 Vickers. However, at high harness the properties may not be desirable. In instances where the properties of fully annealed material is desired, but only the surface requires to be hardened as in this disclosure, the present disclosure includes a method that can provide benefits of both a softer metal alloy with harder outer surface or shell. A non-limiting example is an orthopedic screw where a softer iron alloy is desired for high ductility as well as ease of machinability. Simultaneously, a hard shell is desired of the finished screw. While the inner hardness can range from 250 Vickers to 550 Vickers, the outer harness can vary from 350 Vickers to 1000 Vickers when using alloy (e.g., MoHfC, MoY₂O₃, MoCs₂O, MoW, MoTa, MoZrO₂, MoRe alloy, NiCoCrMo alloy, NiCrMoTi alloy, NiCrCuNb alloy, TiAlV alloy, etc.).

In still yet another and/or alternative non-limiting aspect of the present disclosure, the alloy can be used to form a core of a portion or all of a medical device. For example, a medical device can be in the form of a rod. The core of the rod can be formed of the alloy and then the outside of the core can then be coated with one or more other materials (e.g., another type of metal or alloy, polymer coating, ceramic coating, composite material coating, etc.). Such a rod can be used, for example, for orthopedic applications such as, but not limited to, spinal rods and/or pedicle screw systems. Non-limiting benefits to use the alloy in the core of a medical device can be used to reduce the size of the medical device, increase the strength of the medical device, and/or maintain or reduce the cost of the medical device. As can be appreciated, the alloy can have other or additional advantages. As can also be appreciated, the alloy can form the core of other or additional types of medical devices. The core size and/or thickness of the alloy are non-limiting. In one non-limiting example, there is provided a medical device in the form of a clad rod wherein in the core of the rod is formed of an alloy, and the other layer of the clad rod is formed of a metal or alloy (e.g., MoHfC, MoY₂O₃, MoCs₂O, MoW, MoTa, MoZrO₂, MoRe alloy, NiCoCrMo alloy, NiCrMoTi alloy, NiCrCuNb alloy, TiAlV alloy, etc.). The core and the other layer of the rod can each form 50-99% of the overall cross section of the rod. As can also be appreciated, the alloy can form the core of other or additional types of medical devices.

In another and/or alternative non-limiting aspect of the present disclosure, the alloy is used to form all or a portion of the medical device. In particular, an alloy includes nickel and chromium and one or more alloying agents such as, but are not limited to, aluminum, calcium, carbon, cerium oxide, cobalt, copper, gold, hafnium, iron, lanthanum oxide, lead, magnesium, molybdenum, niobium, osmium, platinum, rare earth metals, rhenium, silver, tantalum, technetium, titanium, tungsten, vanadium, yttrium, yttrium oxide, zinc, zirconium, zirconium oxide, and/or alloys of one or more of such components (e.g., MoHfC, MoY₂O₃, MoCs₂O, MoW, MoTa, MoZrO₂, MoRe alloy, NiCoCrMo alloy, NiCrMoTi alloy, NiCrCuNb alloy, TiAlV alloy, etc.). In one non-limiting formulation, the alloy includes iron and two of more metals selected from the group of nickel, chromium and molybdenum.

In a further and/or alternative non-limiting aspect of the present disclosure, the alloy can be used to form part or all of a medical device in the form of a spinal cage, pedicle screw, and/rod system. One of the most costly health problems in society involves back pain and pathology of the spine. These problems can affect individuals of all ages and can result in great suffering to victims. Back pain can be caused by several factors such as congenital deformities, traumatic injuries, degenerative changes to the spine, and the like. Such changes can cause painful excessive motion, or collapse of a motion segment resulting in the contraction of the spinal canal and compression of the neural structures, causing debilitating pain, paralysis or both, which in tum can result in nerve root compression or spinal stenosis. Nerve conduction disorders can also be associated with intervertebral discs or the vertebrae themselves. One such condition is herniation of the intervertebral disc, in which a small amount of tissue protrudes from the sides of the disc into the foramen to compress the spinal cord. A second common condition involves the development of small bone spurs, termed ‘osteophytes’, along the posterior surface of the vertebral body, again impinging on the spinal cord. Upon identification of these abnormalities, surgery may be required to correct the problem. For those problems associated with the formation of osteophytes or herniations of the intervertebral disc, one such surgical procedure is intervertebral discectomy. In this procedure, the involved vertebrae are exposed and the intervertebral disc is removed, thus removing the offending tissue or providing access for the removal of the bone osteophytes. A second procedure, termed a spinal fusion, may then be required to fix the vertebrae together to prevent movement and maintain a space originally occupied by the intervertebral disc. Although this procedure may result in some minor loss of flexibility in the spine due to the relatively large number of vertebrae, the minor loss of mobility is typically acceptable. For the replacement of a vertebra of the human spinal column, for the distraction of the spinal column, for the stabilization of the vertebrae and likewise, it is known to apply spinal cages and/or pedicle screws. In certain spinal surgeries, a pedicle screw is screwed into the pedicle of the vertebra and the head of the pedicle screw is connected to suitable provisions, for example, to a stabilizing system, to distraction rods, etc. For purposes of the present disclosure, the term ‘pedicle screw’ is intended to cover traditional pedicle screws, nails and posts. It should also be appreciated that the pedicle screw can be used in other applications that do not involve the spine. As such, the pedicle screw can be used in other areas of a body and in many other types of bones. The pedicle screw is generally used to anchor and/or affix an implant (e.g., rod, spinal cage, stabilization system, etc.) to the bone and/or cartilage; however, the pedicle screw can be used for other uses such as, but not limited to, attachment of ligaments; connecting and/or repairing broken bones; reducing pain; stabilizing a tissue ligaments, cartilage, and/or bone; an adjunct for another surgical procedure, and the like. The pedicle screw can be used in areas of a body other than the spine. Such bones in such other areas include, but are not limited to, acromion, atlas, axis, calcaneus, carpus, clavicle, coccyx, epicondyle, epitrochlea, femur, fibula, frontal bone, greater trochanter, humerus, ilium, ischium, mandible, maxilla, metacarpus, metatarsus, occipital bone, olecranon, parietal bone, patella, phalanx, radius, ribs, sacrum, scapula, sternum, talus, tarsus, temporal bone, tibia, ulna, and/or zygomatic bone. The pedicle screw can be used to connect together fractured or broken bones. The bone or bones are not limited to bones of the vertebra, but include any bone in which the pedicle screw can be used to at least partially heal the bone. The pedicle screw can be used to connect ligaments together and/or to bone and/or cartilage. The pedicle screw can be used to retain tissue (e.g., organs, muscle, etc.) in place. Some of these pedicle screw designs are disclosed in U.S. Pat. Nos. 5,882,350; 5,989,254; 5,997,539; 6,004,322; 6,004,349; 6,017,344; 6,053,917; 6,056,753; 6,083,227; 6,113,601; 6,183,472; 6,224,596; 6,368,319; 6,375,657; and 6,402,752; and the patents cited and disclosed in such patents. All these designs of pedicle screws are incorporated herein by reference.

In yet another and/or alternative non-limiting aspect of the present disclosure, the alloy includes a certain amount of carbon and oxygen; however, this is not required. These two elements have been found to affect the forming properties and brittleness of the alloy. The controlled atomic ratio of carbon and oxygen of the alloy also can be used to minimize the tendency of the alloy to form micro-cracks during the forming of the alloy into a medical device, and/or during the use and/or expansion of the medical device in a body passageway. The control of the atomic ratio of carbon to oxygen in the alloy allows for the redistribution of oxygen in the alloy so as to minimize the tendency of micro-cracking in the alloy during the forming of the alloy into a medical device, and/or during the use and/or expansion of the medical device in a body passageway. The atomic ratio of carbon to oxygen in the alloy is believed to be important to minimize the tendency of micro-cracking in the alloy and improve the degree of elongation of the alloy, both of which can affect one or more physical properties of the alloy that are useful or desired in forming and/or using the medical device. The carbon to oxygen atomic ratio can be as low as about 0.2:1. In one non-limiting formulation, the carbon to oxygen atomic ratio in the alloy is generally at least about 0.4:1 (i.e., weight ratio of about 0.3:1). In another non-limiting formulation, the carbon to oxygen atomic ratio in the alloy is generally at least about 0.5:1 (i.e., weight ratio of about 0.375:1). In still another non-limiting formulation, the carbon to oxygen atomic ratio in the alloy is generally at least about 1:1 (i.e., weight ratio of about 0.75:1). In yet another non-limiting formulation, the carbon to oxygen atomic ratio in the alloy is generally at least about 2:1 (i.e., weight ratio of about 1.5:1). Instill yet another non-limiting formulation, the carbon to oxygen atomic ratio in the alloy is generally at least about 2.5:1 (i.e., weight ratio of about 1.88:1). Instill another non-limiting formulation, the carbon to oxygen atomic ratio in the alloy is generally at least about 3:1 (i.e., weight ratio of about 2.25:1). In yet another non-limiting formulation, the carbon to oxygen atomic ratio of the alloy is generally at least about 4:1 (i.e., weight ratio of about 3:1). In still yet another non-limiting formulation, the carbon to oxygen atomic ratio of the alloy is generally at least about 5:1 (i.e., weight ratio of about 3.75:1). In still another non-limiting formulation, the carbon to oxygen atomic ratio in the alloy is generally about 2.5-50:1 (i.e., weight ratio of about 1.88-37.54:1). In a further non-limiting formulation, the carbon to oxygen atomic ratio in the alloy is generally about 2.5-20: 1 (i.e., weight ratio of about 1.88-15:1). In a further non-limiting formulation, the carbon to oxygen atomic ratio in the alloy is generally about 2.5-13.3:1 (i.e., weight ratio of about 1.88-10:1). In still a further non-limiting formulation, the carbon to oxygen atomic ratio in the alloy is generally about 2.5-10:1 (i.e., weight ratio of about 1.88-7.5:1). In yet a further non-limiting formulation, the carbon to oxygen atomic ratio in the alloy is generally about 2.5-5:1 (i.e., weight ratio of about 1.88-3.75:1). As can be appreciated, other atomic ratios of the carbon to oxygen in the alloy can be used. The carbon to oxygen ratio can be adjusted. By intentionally adding carbon to the alloy until the desired carbon to oxygen ratio is obtained. Typically, the carbon content of the alloy is less than about 0.3 weight percent. Carbon contents that are too large can adversely affect the physical properties of the alloy. In one non-limiting formulation, the carbon content of the alloy is less than about 0.1 weight percent of the alloy. In another non-limiting formulation, the carbon content of the alloy is less than about 0.05 weight percent of the alloy of the alloy. In still another non-limiting formulation, the carbon content of the alloy is less than about 0.04 weight percent of the alloy. When carbon is not intentionally added to the alloy of the alloy, the alloy can include up to about 150 ppm carbon, typically up to about 100 ppm carbon, and more typically less than about 50 ppm carbon. The oxygen content of the alloy can vary depending on the processing parameters used to form the alloy of the alloy. Generally, the oxygen content is to be maintained at very low levels. In one non-limiting formulation, the oxygen content is less than about 0.2 weight percent of the alloy. In another non-limiting formulation, the oxygen content is less than about 0.05 weight percent of the alloy. In still another non-limiting formulation, the oxygen content is less than about 0.04 weight percent of the alloy. In yet another non-limiting formulation, the oxygen content is less than about 0.03 weight percent of the alloy. In still yet another non-limiting formulation, the alloy includes up to about 100 ppm oxygen. In a further non-limiting formulation, the alloy includes up to about 75 ppm oxygen. In still a further non-limiting formulation, the alloy includes up to about 50 ppm oxygen. In yet a further non-limiting formulation, the alloy includes up to about 30 ppm oxygen. In still yet a further non- limiting formulation, the alloy includes less than about 20 ppm oxygen. In yet a further non-limiting formulation, the alloy includes less than about 10 ppm oxygen. As can be appreciated, other amounts of carbon and/or oxygen in the alloy can exist. It is believed that the alloy will have a very low tendency to form micro-cracks during the formation of the medical device and after the medical device has been inserted into a patient by closely controlling the carbon to oxygen ration when the oxygen content exceed a certain amount in t the alloy. In one non-limiting arrangement, the carbon to oxygen atomic ratio in the alloy is at least about 2.5:1 when the oxygen content is greater than about 100 ppm in the alloy of the alloy.

In still yet another and/or alternative non-limiting aspect of the present disclosure, the alloy includes a controlled amount of nitrogen; however, this is not required. Large amounts of nitrogen in the alloy can adversely affect the ductility of the alloy of the alloy. This can in tum adversely affect the elongation properties of the alloy. A too-high nitrogen content in the alloy can begin to cause the ductility of the alloy of the alloy to unacceptably decrease, thus adversely affect one or more physical properties of the alloy that are useful or desired in forming and/or using the medical device. In one non-limiting formulation, the alloy includes less than about 0.05 weight percent nitrogen. In another non-limiting formulation, the alloy includes less than about 0.0008 weight percent nitrogen. In still another non-limiting formulation, the alloy includes less than about 0.0004 weight percent nitrogen. In yet another non-limiting formulation, the alloy includes less than about 30 ppm nitrogen. In still yet another non-limiting formulation, the alloy includes less than about 25 ppm nitrogen. In still another non-limiting formulation, the alloy includes less than about 10 ppm nitrogen. In yet another non-limiting formulation, the alloy of the alloy includes less than about 5 ppm nitrogen. As can be appreciated, other amounts of nitrogen in the alloy can exist. The relationship of carbon, oxygen and nitrogen in the alloy is also believed to be important. It is believed that the nitrogen content should be less than the content of carbon or oxygen in the alloy. In one non-limiting formulation, the atomic ratio of carbon to nitrogen is at least about 2:1 (i.e., weight ratio of about 1.71:1). In another non- limiting formulation, the atomic ratio of carbon to nitrogen is at least about 3:1 (i.e., weight ratio of about 2.57:1). In still another non-limiting formulation, the atomic ratio of carbon to nitrogen is about 4-100:1 (i.e., weight ratio of about 3.43-85.7:1). In yet another non-limiting formulation, the atomic ratio of carbon to nitrogen is about 4-75:1 (i.e., weight ratio of about 3.43-64.3:1). In still another non-limiting formulation, the atomic ratio of carbon to nitrogen is about 4-50:1 (i.e., weight ratio of about 3.43-42.85:1). In yet another non-limiting formulation, the atomic ratio of carbon to nitrogen is about 4-35:1 (i.e., weight ratio of about 3.43-30:1). In still yet another non-limiting formulation, the atomic ratio of carbon to nitrogen is about 4-25:1 (i.e., weight ratio of about 3.43-21.43:1). In a further non-limiting formulation, the atomic ratio of oxygen to nitrogen is at least about 1.2:1 (i.e., weight ratio of about 1.37:1). In another non-limiting formulation, the atomic ratio of oxygen to nitrogen is at least about 2:1 (i.e., weight ratio of about 2.28:1). In still another non-limiting formulation, the atomic ratio of oxygen to nitrogen is about 3-100:1 (i.e., weight ratio of about 3.42-114.2:1). In yet another non-limiting formulation, the atomic ratio of oxygen to nitrogen is at least about 3-75:1 (i.e., weight ratio of about 3.42-85.65:1). In still yet another non-limiting formulation, the atomic ratio of oxygen to nitrogen is at least about 3-55:1 (i.e., weight ratio of about 3.42-62.81:1). In yet another non-limiting formulation, the atomic ratio of oxygen to nitrogen is at least about 3-50:1 (i.e., weight ratio of about 3.42-57.1:1).

In a further and/or alternative non-limiting aspect of the present disclosure, the alloy has several physical properties that positively affect the medical device when at least partially formed of the alloy. In one non-limiting embodiment of the disclosure, the average Rockwell A hardness of at least about 30 at 77° F. In one non-limiting aspect of this embodiment, the average hardness of the alloy used to form the medical device is generally at least about 30-62 at 77° F. In another and/or alternative non-limiting embodiment of the disclosure, the average ultimate tensile strength of the alloy used to form the medical device is generally at least about 30 UTS (ksi); however, this is not required. In one non-limiting aspect of this embodiment, the average ultimate tensile strength of the alloy used to form the medical device is generally at least about 35-320 UTS (ksi). In yet another and/or alternative non-limiting embodiment of the disclosure, the average grain size of the alloy used to form the medical device is no greater than about 4 ASTM (e.g., ASTM 112-96); however, this is not required. The grain size as small as about 14-15 ASTM can be achieved; however, the grain size is typically larger than 15 ASTM. The small grain size of the alloy enables the medical device to have the desired elongation and ductility properties that are useful in enabling the medical device to be formed, crimped and/or expanded. In one non-limiting aspect of this embodiment, the average grain size of the alloy used to form the medical device is about 5.2-10 ASTM, typically about 5.5-9 ASTM, more typically about 6-9 ASTM, still more typically about 6-9 ASTM, even more typically about 6.6-9 ASTM, and still even more typically about 7-8.5 ASTM; however, this is not required.

In still yet another and/or alternative non-limiting embodiment of the disclosure, the average tensile elongation of the alloy used to form the medical device is at least about 25%. An average tensile elongation of at least 25% for the alloy is important to enable the medical device to be properly expanded when positioned in the treatment area of a body passageway. A medical device that does not have an average tensile elongation of at least about 25% can form micro-cracks and/or break during the forming, crimping and/or expansion of the medical device. In one non-limiting aspect of this embodiment, the average tensile elongation of the alloy used to form the medical device is about 25-35%. The unique alloy in combination with achieving the desired purity and composition of the alloy and the desired grain size of the alloy results in 1) a medical device having the desired high ductility at about room temperature, 2) a medical device having the desired amount of tensile elongation, 3) a homogeneous or solid solution of an alloy having high radiopacity, 4) a reduction or prevention of micro-crack formation and/or breaking of the alloy tube when the alloy tube is sized and/or cut to form the medical device, 5) a reduction or prevention of micro-crack formation and/or breaking of the medical device when the medical device is crimped onto a balloon and/or other type of medical device for insertion into a body passageway, 6) a reduction or prevention of micro-crack formation and/or breaking of the medical device when the medical device is bent and/or expanded in a body passageway, 7) a medical device having the desired ultimate tensile strength and yield strength, 8) a medical device that can have very thin wall thicknesses and still have the desired radial forces needed to retain the body passageway on an open state when the medical device has been expanded, and/or 9) a medical device that exhibits less recoil when the medical device is crimped onto a delivery system and/or expanded in a body passageway.

In still a further and/or alternative non-limiting aspect of the present disclosure, the alloy is at least partially formed by a swaging process; however, this is not required. In one non-limiting embodiment, the medical device includes one or more rods or tubes upon which swaging is performed to at least partially or fully achieve final dimensions of one or more portions of the medical device. The swaging dies can be shaped to fit the final dimension of the medical device; however, this is not required. Where there are undercuts of hollow structures in the medical device, which is not required, a separate piece of metal can be placed in the undercut to at least partially fill the gap. The separate piece of metal, when used, can be designed to be later removed from the undercut; however, this is not required. The swaging operation can be performed on the medical device in the areas to be hardened. For a round or curved portion of a medical device, the swaging can be rotary. For non-round portion of the medical device, the swaging of the non-round portion of the medical device can be performed by non-rotating swaging dies. The dies can optionally be made to oscillate in radial and/or longitudinal directions instead of or in addition to rotating. The medical device can optionally be swaged in multiple directions in a single operation or in multiple operations to achieve a hardness in desired location and/or direction of the medical device. The swaging temperature for a particular alloy. The swaging process can be conducted by repeatedly hammering the medical device at the location to be hardened at the desired swaging temperature.

Several non-limiting examples of the metal alloy that can be made in accordance with the present disclosure are set forth below. It should be understood, however, that alloys other than those described herein can be used. Other alloys can include, but are not limited to, Ti, Ti alloys, CoCr based alloys, and implantable alloys used in the treatment and/or correction of spine and orthopaedic pathologies.

| Metal/Wt. % | Ex. 1 | Ex. 2 | Ex. 3 |
|---|---|---|---|
| C | <150 ppm | <50 ppm | <50 ppm |
| Mo | 51-54% | 52.5-55.5% | 50.5-52.4% |
| O | <50 ppm | <10 ppm | <10 ppm |
| N | <20 ppm | <10 ppm | <10 ppm |
| Re | 46-49% | 44.5-47.5% | 47.6-49.5% |

| Metal/Wt. % | Ex. 4 | Ex. 5 | Ex. 6 | Ex. 7 |
|---|---|---|---|---|
| C | ≤50 ppm | ≤50 ppm | ≤50 ppm | ≤50 ppm |
| Mo | 51-54% | 52.5-55.5% | 52-56% | 52.5-55% |
| O | ≤20 ppm | ≤20 ppm | ≤10 ppm | ≤10 ppm |
| N | ≤20 ppm | ≤20 ppm | ≤10 ppm | ≤10 ppm |
| Re | 46-49% | 44.5-47.5% | 44-48% | 45-47.5% |
| Ti | ≤0.4% | ≤0.4% | 0.2-0.4% | 0.3-0.4% |
| Y | ≤0.1% | ≤0.1% | 0-0.08% | 0.005-0.05% |
| Zr | ≤0.2% | ≤0.2% | 0-0.2% | 0.1-0.25% |

| Metal/Wt. % | Ex. 8 | Ex. 9 | Ex. 10 | Ex. 11 |
|---|---|---|---|---|
| C | ≤40 ppm | ≤40 ppm | ≤40 ppm | ≤40 ppm |
| Mo | 50.5-53% | 51.5-54% | 52-55% | 52.5-55% |
| O | ≤15 ppm | ≤15 ppm | ≤15 ppm | ≤10 ppm |
| N | ≤10 ppm | ≤10 ppm | ≤10 ppm | ≤10 ppm |
| Re | 47-49.5% | 46-48.5% | 45-48% | 45-47.5% |
| Ti | 0.1-0.35% | 0% | 0% | 0.1-0.3% |
| Y | 0% | 0.002-0.08% | 0% | 0% |
| Zr | 0% | 0% | 00.1-0.2% | 0.05-0.15% |

| Metal/Wt. % | Ex. 12 | Ex. 13 | Ex. 14 | Ex. 15 |
|---|---|---|---|---|
| C | ≤40 ppm | ≤40 ppm | <150 ppm | <150 ppm |
| Mo | 52-55% | 52.5-55.5% | 50-60% | 50-60% |
| O | ≤10 ppm | ≤10 ppm | ≤100 ppm | ≤100 ppm |
| N | ≤10 ppm | ≤10 ppm | ≤40 ppm | ≤40 ppm |
| Re | 45-49% | 44.5-47.5% | 40-50% | 40-50% |
| Ti | 0.05-0.4% | 0% | 0% | 51% |
| Y | 0.005-0.07% | 0.004-0.06% | 0% | ≤0.1% |
| Zr | 0% | 0.1-0.2% | 0% | ≤2% |

| Metal/Wt. % | Ex. 16. | Ex. 17 | Ex. 18 | Ex. 19 |
|---|---|---|---|---|
| C | ≤150 ppm | ≤150 ppm | ≤150 ppm | ≤150 ppm |
| Mo | 50-55% | 52-55.5% | 51-58% | 50-56% |
| O | ≤100 ppm | ≤100 ppm | ≤100 ppm | ≤100 ppm |
| N | ≤40 ppm | ≤20 ppm | ≤20 ppm | ≤20 ppm |
| Re | 45-50% | 44.5-48% | 42-49% | 44-50% |
| Ti | 0% | 0% | 0% | 0% |
| Y | 0% | 0% | 0% | 0% |
| Zr | 0% | 0% | 0% | 0% |

| Metal/Wt. % | Ex. 20 | Ex. 21 | Ex. 22 |
|---|---|---|---|
| C | <150 ppm | <50 ppm | <50 ppm |
| Mo | 51-54% | 52.5-55.5% | 50.5-52.4% |
| O | <50 ppm | <10 ppm | <10 ppm |
| N | <20 ppm | <10 ppm | <10 ppm |
| Re | 46-49% | 44.5-47.5% | 47.6-49.5% |
| Ti | 0% | 0% | 0% |
| Y | 0% | 0% | 0% |
| Zr | 0% | 0% | 0% |

| Metal/Wt. % | Ex. 23 | Ex. 24 | Ex. 25 |
|---|---|---|---|
| C | ≤150 ppm | ≤150 ppm | ≤150 ppm |
| Mo | 50-60% | 50-60% | 50-55% |
| O | ≤100 ppm | ≤100 ppm | ≤100 ppm |
| N | ≤40 ppm | ≤40 ppm | ≤40 ppm |
| Re | 40-50% | 40-50% | 45-50% |
| Ti | ≤0.5% | ≤0.5% | ≤0.5% |
| Y | ≤0.1% | ≤0.1% | ≤0.1% |
| Zr | ≤0.25% | ≤0.25% | ≤0.25% |
| Metal/Wt. % | Ex. 26 | Ex. 27 | Ex. 28 |
| C | ≤150 ppm | ≤150 ppm | ≤150 ppm |
| Mo | 52-55.5% | 51-58% | 50-56% |
| O | ≤100 ppm | ≤100 ppm | ≤100 ppm |
| N | ≤20 ppm | ≤20 ppm | ≤20 ppm |
| Re | 44.5-48% | 42-49% | 44-50% |
| Ti | ≤0.5% | ≤0.5% | ≤0.5% |
| Y | ≤0.1% | ≤0.1% | ≤0.1% |
| Zr | ≤0.25% | ≤0.25% | ≤0.25% |

| Metal/Wt. % | Ex. 29 | Ex. 30 | Ex. 31 | Ex. 32 |
|---|---|---|---|---|
| C | ≤50 ppm | ≤50 ppm | ≤50 ppm | ≤50 ppm |
| Mo | 51-54% | 52.5-55.5% | 52-56% | 52.5-55% |
| O | ≤20 ppm | ≤20 ppm | ≤10 ppm | ≤10 ppm |
| N | ≤20 ppm | ≤20 ppm | ≤10 ppm | ≤10 ppm |
| Re | 46-49% | 44.5-47.5% | 44-48% | 45-47.5% |
| Ti | ≤0.4% | ≤0.4% | 0.2-0.4% | 0.3-0.4% |
| Y | ≤0.1% | ≤0.1% | 0-0.08% | 0.005-0.05% |
| Zr | ≤0.2% | ≤0.2% | 0-0.2% | 0.1-0.25% |

| Metal/Wt. % | Ex. 33 | Ex. 34 | Ex. 35 | Ex. 36 |
|---|---|---|---|---|
| C | ≤40 ppm | ≤40 ppm | ≤40 ppm | ≤40 ppm |
| Mo | 50.5-53% | 51.5-54% | 52-55% | 52.5-55% |
| O | ≤15 ppm | ≤15 ppm | ≤15 ppm | ≤10 ppm |
| N | ≤10 ppm | ≤10 ppm | ≤10 ppm | ≤10 ppm |
| Re | 47-49.5% | 46-48.5% | 45-48% | 45-47.5% |
| Ti | 0.1-0.35% | 0% | 0% | 0.1-0.3% |
| Y | 0% | 0.002-0.08% | 0% | 0% |
| Zr | 0% | 0% | 0.01-0.2% | 0.05-0.15% |

| Metal/Wt. % | Ex. 37 | Ex. 38 |
|---|---|---|
| C | ≤40 ppm | ≤40 ppm |
| Mo | 52-55% | 52.5-55.5% |
| O | ≤10 ppm | ≤10 ppm |
| N | ≤10 ppm | ≤10 ppm |
| Re | 45-49% | 44.5-47.5% |
| Ti | 0.05-0.4% | 0% |
| Y | 0.005-0.07% | 0.004-0.06% |
| Zr | 0% | 0.1-0.2% |

| Metal/Wt. % | Ex. 39 |
|---|---|
| C | ≤150 ppm |
| Mo | 50-60% |
| O | ≤100 ppm |
| N | ≤40 ppm |
| Nb | ≤5% |
| Rare Earth Metal | ≤4% |
| Re | 40-50% |
| Ta | ≤3% |
| Ti | ≤1% |
| W | ≤3% |
| Y | ≤0.1% |
| Zn | ≤0.1% |
| Zr | ≤2% |

| Metal/Wt. % | Ex. 40 |
|---|---|
| C | ≤0.01% |
| Co | ≤0.002% |
| Fe | ≤0.02% |
| H | ≤0.002% |
| Mo | 52-53% |
| N | ≤0.0008% |
| Ni | ≤0.01% |
| O | ≤0.06% |
| Re | 47-48% |
| S | ≤0.008% |
| Sn | ≤0.002% |
| Ti | ≤0.002% |
| W | ≤0.02% |

| Metal/Wt. % | Ex. 41 | Ex. 42 | Ex. 43 | Ex. 44 |
|---|---|---|---|---|
| C | 0-50 ppm | 0-50 ppm | 0-50 ppm | 0-50 ppm |
| Ca | 0-1% | 0-0.5% | 0% | 0% |
| Mg | 0% | 0-3% | 0% | 0% |
| Mo | 0% | 0-2% | 0% | 0% |
| O | 0-50 ppm | 0-50 ppm | 0-50 ppm | 0-50 ppm |
| N | 0-50 ppm | 0-50 ppm | 0-50 ppm | 0-50 ppm |
| Rare Earth Metal | 0-1% | 0-0.5% | 0% | 0% |
| Re | 0-6% | 0-5% | 0-4% | 0% |
| Ta | 85-96% | 10-90% | 85-95% | 90.5-98% |
| W | 4-15% | 10-90% | 5-15% | 2-9.5% |
| Y | 0% | 0-1% | 0% | 0% |
| Zn | 0% | 0-1% | 0% | 0% |
| Zr | 0% | 0-1% | 0% | 0% |

| Metal/Wt. % | Ex. 45 | Ex. 46 |
|---|---|---|
| C | 0-50 ppm | 0-50 ppm |
| Ca | 0% | 0% |
| Mg | 0% | 0% |
| Mo | 0% | 0% |
| O | 0-50 ppm | 0-50 ppm |
| N | 0-50 ppm | 0-50 ppm |
| Rare Earth Metal | 0% | 0% |
| Re | 0-4% | 0% |
| Ta | 95-98% | 90-97.5% |
| W | 2% to less than 5% | 2.5-10% |
| Y | 0% | 0% |
| Zn | 0% | 0% |
| Zr | 0% | 0% |

In Examples 1-3, 14, 16-19, and 20-22 above, it will be appreciated that all of the above ranges include values between the range and other ranges that are between the range as set forth above. The metal alloy is principally formed of rhenium and molybdenum and the content of other metals and/or impurities is less than about 0.1 weight percent of the metal alloy, the atomic ratio of carbon to oxygen is about 2.5-10: 1 (i.e., weight ratio of about 1.88- 7.5:1), the average grain size of the metal alloy is about 6-10 ASTM, the tensile elongation of the metal alloy is about 25-35%, the average density of the metal alloy is at least about 13.4 gm/cc, the average yield strength of the metal alloy is about 98-122 (ksi), the average ultimate tensile strength of the metal alloy is about 150-310 UTS (ksi), and an average Vickers hardness of 372-653 (i.e., Rockwell A Hardness of about 70-80 at 77° F., an average Rockwell C Hardness of about 39-58 at 77° F.). In Examples 4-7, 8-11, 12, 13, 15, and 32-38 above, the metal alloy is principally formed of rhenium and molybdenum and at least one metal of titanium, yttrium and/or zirconium, and the content of other metals and/or impurities is less than about 0.1 weight percent of the metal alloy, the ratio of carbon to oxygen is about 2.5-10: 1, the average grain size of the metal alloy is about 6-10 ASTM, the tensile elongation of the metal alloy is about 25-35%, the average density of the metal alloy is at least about 13.6 gm/cc, the average yield strength of the metal alloy is at least about 110 (ksi), the average ultimate tensile strength of the metal alloy is about 150-310 UTS (ksi), and an average Vickers hardness of 372-653 (i.e., an average Rockwell A Hardness of about 70-80 at 77° F., an average Rockwell C Hardness of about 39-58 at 77° F.). The remaining alloys identified in the above examples may or may not include titanium, yttrium and/or zirconium. The properties of these alloys will be similar to the alloys discussed in the above examples. In Example 32, the weight ratio of titanium to zirconium is about 1.5-3:1. In Example 36, the weight ratio of titanium to zirconium is about 1.75-2.5:1. In Examples 29-32, the weight ratio of titanium to zirconium is about 1-10:1. In Example 40, the ratio of carbon to oxygen is at least about 0.4:1 (i.e., weight ratio of carbon to oxygen of at least about 0.3:1), the nitrogen content is less than the carbon content and the oxygen content, the atomic ratio of carbon to nitrogen is at least about 4:1 (i.e., weight ratio of about 3.43:1), the atomic ratio of oxygen to nitrogen is at least about 3:1 (i.e., weight ratio of about 3.42:1), the average grain size of metal alloy is about 6-10 ASTM, the tensile elongation of the metal alloy is about 25- 35%, the average density of the metal alloy is at least about 13.4 gm/cc, the average yield strength of the metal alloy is about 98-122 (ksi), the average ultimate tensile strength of the metal alloy is about 100-150 UTS (ksi), and the average hardness of the metal alloy is about 80-100 (HRC) at 77° F.

In Examples 41-46, it will be appreciated that all of the above ranges include and value between the range and other range that is between the range as set forth above. The metal alloy is principally formed of tungsten and tantalum and the content of other metals and/or impurities is less than about 0.1 weight percent, and typically less than 0.04 weight percent of the metal alloy.

| Metal/Wt. % | Ex. 47 | Ex. 48 | Ex. 49 |
|---|---|---|---|
| C | <150 ppm | <50 ppm | <50 ppm |
| Mo | 51-54% | 52.5-55.5% | 50.5-52.4% |
| O | <50 ppm | <10 ppm | <10 ppm |
| N | <20 ppm | <10 ppm | <10 ppm |
| Re | 46-49% | 44.5-47.5% | 47.6-49.5% |
| CNT | 0.05-10% | 0.05-10% | 0.05-10% |

| Metal/Wt % | Ex. 50 | Ex. 51 | Ex. 52 | Ex. 53 |
|---|---|---|---|---|
| C | ≤50 ppm | ≤50 ppm | ≤50 ppm | ≤50 ppm |
| Mo | 51-54% | 52.5-55.5% | 52-56% | 52.5-55% |
| O | ≤20 ppm | ≤20 ppm | ≤10 ppm | ≤10 ppm |
| N | ≤20 ppm | ≤20 ppm | ≤10 ppm | ≤10 ppm |
| Re | 46-49% | 44.5-47.5% | 44-48% | 45-47.5% |
| CNT | 0.1-8% | 0.1-8% | 0.1-8% | 0.1-8% |

| Metal/Wt. % | Ex. 54 | Ex. 55 | Ex. 56 | Ex. 57 |
|---|---|---|---|---|
| C | ≤40 ppm | ≤40 ppm | ≤40 ppm | ≤40 ppm |
| Mo | 50.5-53% | 51.5-54% | 52-55% | 52.5-55% |
| O | ≤15 ppm | ≤15 ppm | ≤15 ppm | ≤10 ppm |
| N | ≤10 ppm | ≤10 ppm | ≤10 ppm | ≤10 ppm |
| Re | 47-49.5% | 46-48.5% | 45-48% | 45-47.5% |
| CNT | 0.1-8% | 0.1-8% | 0.1-8% | 0.1-8% |

| Metal/Wt. % | Ex. 58 | Ex. 59 | Ex. 60 | Ex. 61 |
|---|---|---|---|---|
| C | ≤40 ppm | ≤40 ppm | <150 ppm | <150 ppm |
| Mo | 52-55% | 52.5-55.5% | 50-60% | 50-60% |
| O | ≤10 ppm | ≤10 ppm | ≤100 ppm | ≤100 ppm |
| N | ≤10 ppm | ≤10 ppm | ≤40 ppm | ≤40 ppm |
| Re | 45-49% | 44.5-47.3% | 40-50% | 40-50% |
| CNT | 0.1-8% | 0.1-8% | 0.1-8% | 0.1-8% |

| Metal/Wt. % | Ex. 62 | Ex. 63 | Ex. 64 | Ex. 65 |
|---|---|---|---|---|
| C | ≤150 ppm | ≤150 ppm | ≤150 ppm | ≤150 ppm |
| Mo | 50-55% | 52-55.5% | 51-58% | 50-56% |
| O | ≤100 ppm | ≤100 ppm | ≤100 ppm | ≤100 ppm |
| N | ≤40 ppm | ≤20 ppm | ≤20 ppm | ≤20 ppm |
| Re | 45-50% | 44.5-48% | 42-49% | 44-50% |
| CNT | 0.1-8% | 0.1-8% | 0.1-8% | 0.1-8% |

| Metal/Wt. % | Ex. 66 | Ex. 67 | Ex. 68 |
|---|---|---|---|
| C | <150 ppm | <50 ppm | <50 ppm |
| Mo | 51-54% | 52.5-55.5% | 50.5-52.4% |
| O | <50 ppm | <10 ppm | <10 ppm |
| N | <20 ppm | <10 ppm | <10 ppm |
| Re | 46-49% | 44.5-47.5% | 47.6-49.5% |
| CNT | 0.1-8% | 0.1-8% | 0.1-8% |

| Metal/Wt. % | Ex. 69 | Ex. 70 | Ex. 71 |
|---|---|---|---|
| C | ≤150 ppm | ≤150 ppm | ≤150 ppm |
| Mo | 50-60% | 50-60% | 50-55% |
| O | ≤100 ppm | ≤100 ppm | ≤100 ppm |
| N | ≤40 ppm | ≤40 ppm | ≤40 ppm |
| Re | 40-50% | 40-50% | 45-50% |
| CNT | 0.5-5% | 0.5-5% | 0.5-5% |
| Metal/Wt. % | Ex. 72 | Ex. 73 | Ex. 74 |
| C | ≤150 ppm | ≤150 ppm | ≤150 ppm |
| Mo | 52-55.5% | 51-58% | 50-56% |
| O | ≤100 ppm | ≤100 ppm | ≤100 ppm |
| N | ≤20 ppm | ≤20 ppm | ≤20 ppm |
| Re | 44.5-48% | 42-49% | 44-50% |
| CNT | 0.5-5% | 0.5-5% | 0.5-5% |

| Metal/Wt. % | Ex. 75 | Ex. 76 | Ex. 77 | Ex. 78 |
|---|---|---|---|---|
| C | ≤50 ppm | ≤50 ppm | ≤50 ppm | ≤50 ppm |
| Mo | 51-54% | 52.5-55.5% | 52-56% | 52.5-55% |
| O | ≤20 ppm | ≤20 ppm | ≤10 ppm | ≤10 ppm |
| N | ≤20 ppm | ≤20 ppm | ≤10 ppm | ≤10 ppm |
| Re | 46-49% | 44.5-47.5% | 44-48% | 45-47.5% |
| CNT | 0.5-5% | 0.5-5% | 0.5-5% | 0.5-5% |

| Metal/Wt. % | Ex. 79 | Ex. 80 | Ex. 81 | Ex. 82 |
|---|---|---|---|---|
| C | ≤40 ppm | ≤40 ppm | ≤40 ppm | ≤40 ppm |
| Mo | 50.5-53% | 51.5-54% | 52-55% | 52.5-55% |
| O | ≤15 ppm | ≤15 ppm | ≤15 ppm | ≤10 ppm |
| N | ≤10 ppm | ≤10 ppm | ≤10 ppm | ≤10 ppm |
| Re | 47-49.5% | 46-48.5% | 45-48% | 45-47.5% |
| CNT | 0.5-5% | 0.5-5% | 0.5-5% | 0.5-5% |

| Metal/Wt. % | Ex. 83 | Ex. 84 |
|---|---|---|
| C | ≤40 ppm | ≤40 ppm |
| Mo | 52-55% | 52.5-55.5% |
| O | ≤10 ppm | ≤10 ppm |
| N | ≤10 ppm | ≤10 ppm |
| Re | 45-49% | 44.5-47.5% |
| CNT | 0.5-5% | 0.5-5% |

| Metal/Wt. % | Ex. 85 |
|---|---|
| C | ≤150 ppm |
| Hf | ≤5% |
| Mo | 20-90% |
| O | ≤100 ppm |
| Os | ≤5% |
| N | ≤40 ppm |
| Nb | ≤5% |
| Pt | ≤5% |
| Rare Earth Metal | ≤4% |
| Re | 10-80% |
| Ta | ≤3% |
| Tc | ≤5% |
| Ti | ≤1% |
| V | ≤5% |
| W | ≤3% |
| Y | ≤0.1% |
| Zn | ≤0.1% |
| Zr | ≤5% |
| CNT | 0.05-10% |

| Metal/Wt. % | Ex. 86 |
|---|---|
| C | ≤0.01% |
| Co | ≤0.002% |
| Fe | ≤0.02% |
| H | ≤0.002% |
| Hf | ≤1% |
| Mo | 40-90% |
| N | ≤0.0008% |
| Nb | ≤1% |
| Ni | ≤0.01% |
| O | ≤0.06% |
| Os | ≤1% |
| Pt | ≤1% |
| Re | 10-60% |
| S | ≤0.008% |
| Sn | ≤0.002% |
| Tc | ≤1% |
| Ti | ≤1% |
| V | ≤1% |
| W | ≤1% |
| Zr | ≤1% |
| CNT | 0.5-5% |

| Metal/Wt. % | Ex. 87 |
|---|---|
| C | ≤150 ppm |
| Hf | ≤5% |
| Mo | 20-80% |
| O | ≤100 ppm |
| Os | ≤5% |
| N | ≤40 ppm |
| Nb | ≤5% |
| Pt | ≤5% |
| Rare Earth Metal | ≤4% |
| Re | 20-80% |
| Ta | ≤3% |
| Tc | ≤5% |
| Ti | ≤1% |
| V | ≤5% |
| W | ≤3% |
| Y | ≤0.1% |
| Zn | ≤0.1% |
| Zr | ≤5% |

| Metal/Wt. % | Ex. 88 |
|---|---|
| C | ≤0.01% |
| Co | ≤0.002% |
| Fe | ≤0.02% |
| H | ≤0.002% |
| Hf | ≤1% |
| Mo | 40-60% |
| N | ≤0.0008% |
| Nb | ≤1% |
| Ni | ≤0.01% |
| O | ≤0.06% |
| Os | ≤1% |
| Pt | ≤1% |
| Re | 40-60% |
| S | ≤0.008% |
| Sn | ≤0.002% |
| Tc | ≤1% |
| Ti | ≤1% |
| V | ≤1% |
| W | ≤1% |
| Zr | ≤1% |

| Metal/Wt. % | Ex. 89 | Ex. 90 | Ex. 91 | Ex. 92 |
|---|---|---|---|---|
| Mo | 40-99.89% | 40-99.9% | 40-99.89% | 40-99.5% |
| C | 0.01-0.3% | 0-0.3% | 0-0.3% | 0-0.3% |
| Co | ≤0.002% | ≤0.002% | ≤0.002% | ≤0.002% |
| Cs₂O | 0-0.2% | 0-0.2% | 0.01-0.2% | 0-0.2% |
| Fe | ≤0.02% | ≤0.02% | ≤0.02% | ≤0.02% |
| H | ≤0.002% | ≤0.002% | ≤0.002% | ≤0.002% |
| Hf | 0.1-2.5% | 0-2.5% | 0-2.5% | 0-2.5% |
| O | ≤0.06% | ≤0.06% | ≤0.06% | ≤0.06% |
| Os | ≤1% | ≤1% | ≤1% | ≤1% |
| La₂O₃ | 0-2% | 0.1-2% | 0-2% | 0-2% |
| N | ≤20 ppm | ≤20 ppm | ≤20 ppm | ≤20 ppm |
| Nb | ≤0.01% | ≤0.01% | ≤0.01% | ≤0.01% |
| Pt | ≤1% | ≤1% | ≤1% | ≤1% |
| Re | 0-40% | 0-40% | 0-40% | 0-40% |
| S | ≤0.008% | ≤0.008% | ≤0.008% | ≤0.008% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| W | 0-50% | 0-50% | 0-50% | 0.5-50% |
| Y₂O₃ | 0-1% | 0-1% | 0.1-1% | 0-1% |
| Zr | ≤1% | ≤1% | ≤1% | ≤1% |
| ZrO₂ | 0-3% | 0-3% | 0-3% | 0-3% |
| CNT | 0-10% | 0-10% | 0-10% | 0-10% |

| Metal/Wt. % | Ex. 93 | Ex. 94 | Ex. 95 |
|---|---|---|---|
| Mo | 40-99.9% | 40-99.5% | 40-99.5% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 0-40% | 0-40% | 0.5-40% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| W | 0-50% | 0-50% | 0-50% |
| Y₂O₃ | 0-1% | 0-1% | 0-1% |
| ZrO₂ | 0.1-3% | 0-3% | 0-3% |
| CNT | 0-10% | 0-10% | 0-10% |

| Metal/Wt. % | Ex. 96 | Ex. 97 | Ex. 98 | Ex. 99 |
|---|---|---|---|---|
| Mo | 98-99.15% | 98-99.7% | 50-99.66% | 40-80% |
| C | 0.05-0.15% | 0-0.15% | 0-0.15% | 0-0.15% |
| Cs₂O | 0-0.2% | 0-0.2% | 0.04-0.1% | 0-0.2% |
| Hf | 0.8-1.4% | 0-2.5% | 0-2.5% | 0-2.5% |
| La₂O₃ | 0-2% | 0.3-0.7% | 0-2% | 0-2% |
| Re | 0-40% | 0-40% | 0-40% | 0-40% |
| Ta | 0-50% | 0-50% | 0-50% | 0-50% |
| W | 0-50% | 0-50% | 0-50% | 20-50% |
| Y₂O₃ | 0-1% | 0-1% | 0.3-0.5% | 0-1% |
| ZrO₂ | 0-3% | 0-3% | 0-3% | 0-3% |

| Metal/Wt % | Ex. 100 | Ex. 101 | Ex. 102 |
|---|---|---|---|
| Mo | 97-98.8% | 50-90% | 60-99.5% |
| C | 0-0.15% | 0-0.15% | 0-0.15% |
| Cs₂O | 0-0.2% | 0-0.2% | 0-0.2% |
| Hf | 0-2.5% | 0-2.5% | 0-2.5% |
| La₂O₃ | 0-2% | 0-2% | 0-2% |
| Re | 0-40% | 0-40% | 5-40% |
| Ta | 0-50% | 10-50% | 0-50% |
| W | 0-50% | 0-50% | 0-50% |
| Y₂O₃ | 0-1% | 0-1% | 0-1% |
| ZrO₂ | 1.2-1.8% | 0-3% | 0-3% |

| Metal/Wt. % | Ex. 103 | Ex. 104 | Ex. 105 | Ex. 106 |
|---|---|---|---|---|
| Fe | 65-80% | 65-85% | 65-85% | 0-2% |
| Al | 0-7% | 0-7% | 0-7% | 2-9% |
| C | 0.05-0.5% | 0-0.05% | 0-0.15% | 0-0.15% |
| Co | 5-20% | 0-5% | 0-5% | 0-5% |
| Cr | 1-5% | 4-15% | 7-22% | 0-4% |
| Cu | 0-8% | 0-2% | 1-5% | 0-2% |
| Mo | 0.5-4% | 0.3-4% | 0-2% | 0-2% |
| Nb | 0-2% | 0-2% | 0.05-1% | 0-2% |
| Ni | 4-20% | 4-20% | 2-8% | 0-2% |
| Ti | 0-3% | 0.5-4% | 0-3% | 80-91% |
| V | 0-7% | 0-3% | 0-3% | 2-6% |

In Examples 89-106, it will be appreciated that all of the above ranges include and value between the range and other ranges that are between the range as set forth above. In the above metal alloys, the average grain size of the metal alloy can be about 6-10 ASTM, the tensile elongation of the metal alloy can be about 25-35%, the average density of the metal alloy can be at least about 13.4 gm/cc, the average yield strength of the metal alloy can be about 98-122 (ksi), the average ultimate tensile strength of the metal alloy can be about 100- 310 UTS (ksi), an average Vickers hardness of 372-653 (i.e., Rockwell A Hardness can be about 70-100 at 77° F., an average Rockwell C Hardness can be about 39-58 at 77° F., the primarily tensile strength is over 1000 MPa, elongation is >10%; and modulus of elasticity is >300 GPa; however, this is not required.

In the examples above, the atomic ratio of carbon to oxygen can be about 2.5-10:1 (i.e., weight ratio of about 1.88-7.5:1), the average grain size of the alloy can be about 6-10 ASTM, the tensile elongation of the alloy can be about 25-35%, the average density of the alloy can be at least about 13.4 gm/cc, the average yield strength of the alloy can be about 98-122 (ksi), the average ultimate tensile strength of the alloy can be about 150-310 UTS (ksi), and an average Vickers hardness can be 372-653 (i.e., Rockwell A Hardness of about 70-80 at 77° F., an average Rockwell C Hardness of about 39-58 at 77° F.).

Several additional non-limiting examples of the metal alloy that can be made in accordance with the present disclosure are set forth below. As noted above, it should be understood, however, that alloys other than those described herein can be used. Other alloys can include, but are not limited to, Ti, Ti alloys, CoCr based alloys, and implantable alloys used in the treatment and/or correction of spine and orthopaedic pathologies.

| Metal/Wt. % | Ex. 107 | Ex. 108 | Ex. 109 | Ex. 110 |
|---|---|---|---|---|
| Mo | 40-99.89% | 40-99.9% | 40-99.89% | 40-99.5% |
| C | 0.01-0.3% | 0-0.3% | 0-0.3% | 0-0.3% |
| Co | ≤0.002% | ≤0.002% | ≤0.002% | ≤0.002% |
| Cs₂O | 0-0.2% | 0-0.2% | 0.01-0.2% | 0-0.2% |
| Fe | ≤0.02% | ≤0.02% | ≤0.02% | ≤0.02% |
| H | ≤0.002% | ≤0.002% | ≤0.002% | ≤0.002% |
| Hf | 0.1-2.5% | 0-2.5% | 0-2.5% | 0-2.5% |
| O | ≤0.06% | ≤0.06% | ≤0.06% | ≤0.06% |
| Os | ≤1% | ≤1% | ≤1% | ≤1% |
| La₂O₃ | 0-2% | 0.1-2% | 0-2% | 0-2% |
| N | ≤20 ppm | ≤20 ppm | ≤20 ppm | ≤20 ppm |
| Nb | ≤0.01% | ≤0.01% | ≤0.01% | ≤0.01% |
| Pt | ≤1% | ≤1% | ≤1% | ≤1% |
| Re | 0-40% | 0-40% | 0-40% | 0-40% |
| S | ≤0.008% | ≤0.008% | ≤0.008% | ≤0.008% |
| Sn | ≤0.002% | ≤0.002% | ≤0.002% | ≤0.002% |
| Ta | 0-50% | 0-50% | 0-50% | 0-50% |
| Tc | ≤1% | ≤1% | ≤1% | ≤1% |
| Ti | ≤1% | ≤1% | ≤1% | ≤1% |
| V | ≤1% | ≤1% | ≤1% | ≤1% |
| W | 0-50% | 0-50% | 0-50% | 0.5-50% |
| Y₂O₃ | 0-1% | 0-1% | 0.1-1% | 0-1% |
| Zr | ≤1% | ≤1% | ≤1% | ≤1% |
| ZrO₂ | 0-3% | 0-3% | 0-3% | 0-3% |
| CNT | 0-10% | 0-10% | 0-10% | 0-10% |

| Metal/Wt % | Ex. 111 | Ex. 112 | Ex. 113 |
|---|---|---|---|
| Mo | 40-99.9% | 40-99.5% | 40-99.5% |
| C | 0-0.3% | 0-0.3% | 0-0.3% |
| Co | ≤0.002% | ≤0.002% | ≤0.002% |
| Cs₂O | 0-0.2% | 0-0.2% | 0-0.2% |
| H | ≤0.002% | ≤0.002% | ≤0.002% |
| Hf | 0-2.5% | 0-2.5% | 0-2.5% |
| O | ≤0.06% | ≤0.06% | ≤0.06% |
| Os | ≤1% | ≤1% | ≤1% |
| La₂O₃ | 0-2% | 0-2% | 0-2% |
| N | ≤20 ppm | ≤20 ppm | ≤20 ppm |
| Nb | ≤0.01% | ≤0.01% | ≤0.01% |
| Pt | ≤1% | ≤1% | ≤1% |
| Re | 0-40% | 0-40% | 0.5-40% |
| S | ≤0.008% | ≤0.008% | ≤0.008% |
| Sn | ≤0.002% | ≤0.002% | ≤0.002% |
| Ta | 0-50% | 0.5-50% | 0-50% |
| Tc | ≤1% | ≤1% | ≤1% |
| Ti | ≤1% | ≤1% | ≤1% |
| V | ≤1% | ≤1% | ≤1% |
| W | 0-50% | 0-50% | 0-50% |
| Y₂O₃ | 0-1% | 0-1% | 0-1% |
| ZrO₂ | 0.1-3% | 0-3% | 0-3% |
| CNT | 0-10% | 0-10% | 0-10% |

| Metal/Wt. % | Ex. 114 | Ex. 115 | Ex. 116 | Ex. 117 |
|---|---|---|---|---|
| Mo | 98-99.15% | 98-99.7% | 50-99.66% | 40-80% |
| C | 0.05-0.15% | 0-0.15% | 0-0.15% | 0-0.15% |
| Cs₂O | 0-0.2% | 0-0.2% | 0.04-0.1% | 0-0.2% |
| Hf | 0.8-1.4% | 0-2.5% | 0-2.5% | 0-2.5% |
| La₂O₃ | 0-2% | 0.3-0.7% | 0-2% | 0-2% |
| Re | 0-40% | 0-40% | 0-40% | 0-40% |
| Ta | 0-50% | 0-50% | 0-50% | 0-50% |
| W | 0-50% | 0-50% | 0-50% | 20-50% |
| Y₂O₃ | 0-1% | 0-1% | 0.3-0.5% | 0-1% |
| ZrO₂ | 0-3% | 0-3% | 0-3% | 0-3% |

| Metal/Wt. % | Ex. 118 | Ex. 119 | Ex. 120 |
|---|---|---|---|
| Mo | 97-98.8% | 50-90% | 60-99.5% |
| C | 0-0.15% | 0-0.15% | 0-0.15% |
| Cs₂O | 0-0.2% | 0-0.2% | 0-0.2% |
| Hf | 0-2.5% | 0-2.5% | 0-2.5% |
| La₂O₃ | 0-2% | 0-2% | 0-2% |
| Re | 0-40% | 0-40% | 5-40% |
| Ta | 0-50% | 10-50% | 0-50% |
| W | 0-50% | 0-50% | 0-50% |
| Y₂O₃ | 0-1% | 0-1% | 0-1% |
| ZrO₂ | 1.2-1.8% | 0-3% | 0-3% |

| Metal/Wt. % | Ex. 121 | Ex. 122 | Ex. 123 | Ex. 124 |
|---|---|---|---|---|
| Fe | 65-80% | 65-85% | 65-85% | 0-2% |
| Al | 0-7% | 0-7% | 0-7% | 2-9% |
| C | 0.05-0.5% | 0-0.05% | 0-0.15% | 0-0.15% |
| Co | 5-20% | 0-5% | 0-5% | 0-5% |
| Cr | 1-5% | 4-15% | 7-22% | 0-4% |
| Cu | 0-8% | 0-2% | 1-5% | 0-2% |
| Mo | 0.5-4% | 0.3-4% | 0-2% | 0-2% |
| Nb | 0-2% | 0-2% | 0.05-1% | 0-2% |
| Ni | 4-20% | 4-20% | 2-8% | 0-2% |
| Ti | 0-3% | 0.5-4% | 0-3% | 80-91% |
| V | 0-7% | 0-3% | 0-3% | 2-6% |

In Examples 107-124, it will be appreciated that all of the above ranges include and value between the range and other ranges that are between the range as set forth above. In the above metal alloys, the average grain size of the metal alloy can be about 6-10 ASTM, the tensile elongation of the metal alloy can be about 25-35%, the average density of the metal alloy can be at least about 13.4 gm/cc, the average yield strength of the metal alloy can be about 98-122 (ksi), the average ultimate tensile strength of the metal alloy can be about 100- 310 UTS (ksi), an average Vickers hardness of 372-653 (i.e., Rockwell A Hardness can be about 70-100 at 77° F., an average Rockwell C Hardness can be about 39-58 at 77° F., the primarily tensile strength is over 1000 MPa, elongation is >10%; and modulus of elasticity is >300 GPa; however, this is not required.

Non-limiting specific alloys representative of the alloys of Examples 121-124 are as follows:

| | | | | |
|---|---|---|---|---|
| Carbon | 0.21/0.25% | 0.02% Max. | 0.0 [symbol] % Max. | 0.0 [symbol] % Max. |
| Nickel | 11.00/12.00% | 10.75/11.25% | 3.00/5.00% | |
| Cobalt | 13.00/14.00% | | | |
| Chromium | 2.90/ [symbol] 30% | 11.00/12.50% | 15.00/17.50% | |
| Molybdenum | 1.10/1. [symbol] 0% | 0.75/1.25% | | |
| Titanium | | 1.50/1. [symbol] 0% | | Balance |
| Copper | | | 3.00 [symbol] 00% | |
| Niobium | | | 0.150 [symbol] 5% | |
| Aluminum | | | | [symbol] |
| V [symbol] na [symbol] um | | | | [symbol] |
| Nitrogen | | | | 0.05% Max |
| Hydrogen | | | | 0.015% Max. |
| Oxygen | | | | 0.2% Max. |
| Iron | Balance | Balance | Balance | 0.3% Max. |

[symbol]

indicates data missing or illegible when filed

| | | | | | |
|---|---|---|---|---|---|
| U.T.S. MPa | 20 [symbol] 3 | 1795 | 13 [symbol] 5 | [symbol] 95 | 1140 |
| (k [symbol] ) | (302) | (2 [symbol] 0) | (1 [symbol] ) | (130) | (1 [symbol] 5) |
| Y.S. MPa | 17 [symbol] 0 | 1555 | 1 [symbol] 0 | [symbol] 30 | 1035 |
| (k [symbol] ) | (25 [symbol] ) | (240) | (183) | (120) | (150) |
| Elong. % | 14 | 13 | 15 | 10 | 10 |
| R.A. % | 64 | 58 | 52 | 25 | 20 |
| Fracture Toughness | | | | | |
| MPa (m [symbol] ) | 109 | [symbol] 3 | [symbol] 1 | 77 | 39 |
| ( [symbol] )) | (99) | (75) | (5 [symbol] ) | (70) | (35) |
| Density [symbol] | 7 [symbol] 0 | 7 [symbol] 33 | 7 [symbol] | 44 [symbol] | 4429 |
| (lb/in. [symbol] ) | (0.295) | (0.290) | (0.292) | (0.160) | (0.1 [symbol] 0) |
| Modulus of Elasticity | | | | | |
| Gpa | 1 [symbol] 5 | 1 [symbol] | 1 [symbol] .5 | 110. [symbol] | 113. [symbol] |
| (10 [symbol] k [symbol] ) | (27. [symbol] × 10 [symbol] ) | (28.8 × 10 [symbol] ) | (26.5 × 10 [symbol] ) | (10.0 × 10 [symbol] ) | (16.5 × 10 [symbol] ) |
| Strength-to-Density Ratio | | | | | |
| [symbol] | 2 [symbol] | [symbol] 3.3 | 17. [symbol] | 20.7 | 2 [symbol] 2 |
| (10 [symbol] ) | (1060) | (919) | (70 [symbol] ) | ( [symbol] 13) | (10 [symbol] 1) |
| Hardness HRC | 54 | 51 | 44 | 30 | 40 |

[symbol]

indicates data missing or illegible when filed

In yet another and/or alternative non-limiting aspect of the present disclosure, the medical device can include, contain and/or be coated with one or more agents that facilitate in the success of the medical device and/or treated area. The term “agent” includes, but is not limited to a substance, pharmaceutical, biologic, veterinary product, drug, and analogs or derivatives otherwise formulated and/or designed to prevent, inhibit and/or treat one or more clinical and/or biological events, and/or to promote healing. Non-limiting examples of clinical events that can be addressed by one or more agents include, but are not limited to viral, fungus and/or bacterial infection; vascular diseases and/or disorders; digestive diseases and/or disorders; reproductive diseases and/or disorders; lymphatic diseases and/or disorders; cancer; implant rejection; pain; nausea; swelling; arthritis; bone diseases and/or disorders; organ failure; immunity diseases and/or disorders; cholesterol problems; blood diseases and/or disorders; lung diseases and/or disorders; heart diseases and/or disorders; brain diseases and/or disorders; neuralgia diseases and/or disorders; kidney diseases and/or disorders; ulcers; liver diseases and/or disorders; intestinal diseases and/or disorders; gallbladder diseases and/or disorders; pancreatic diseases and/or disorders; psychological disorders; respiratory diseases and/or disorders; gland diseases and/or disorders; skin diseases and/or disorders; hearing diseases and/or disorders; oral diseases and/or disorders; nasal diseases and/or disorders; eye diseases and/or disorders; fatigue; genetic diseases and/or disorders; bums; scarring and/or scars; trauma; weight diseases and/or disorders; addiction diseases and/or disorders; hair loss; cramps; muscle spasms; tissue repair; nerve repair; neural regeneration and/or the like. Non-limiting examples of agents that can be used include, but are not limited to, 5-fluorouracil and/or derivatives thereof; 5-phenylmethimazole and/or derivatives thereof; ACE inhibitors and/or derivatives thereof; acenocoumarol and/or derivatives thereof; acyclovir and/or derivatives thereof; actilyse and/or derivatives thereof; adrenocorticotropic hormone and/or derivatives thereof; adriamycin and/or derivatives thereof; agents that modulate intracellular Ca2+ transport such as L-type (e.g., diltiazem, nifedipine, verapamil, etc.) or T-type Ca2+ channel blockers (e.g., amiloride, etc.); alpha-adrenergic blocking agents and/or derivatives thereof; alteplase and/or derivatives thereof; amino glycosides and/or derivatives thereof (e.g., gentamycin, tobramycin, etc.); angiopeptin and/or derivatives thereof; angiostatic steroid and/or derivatives thereof; angiotensin II receptor antagonists and/or derivatives thereof; anistreplase and/or derivatives thereof; antagonists of vascular epithelial growth factor and/or derivatives thereof; antibiotics; anti- coagulant compounds and/or derivatives thereof; anti-fibrosis compounds and/or derivatives thereof; antifungal compounds and/or derivatives thereof; anti-inflammatory compounds and/or derivatives thereof; anti-invasive factor and/or derivatives thereof; anti-metabolite compounds and/or derivatives thereof (e.g., staurosporin, trichothecenes, and modified diphtheria and ricin toxins, pseudomonas exotoxin, etc.); anti-matrix compounds and/or derivatives thereof (e.g., colchicine, tamoxifen, etc.); anti-microbial agents and/or derivatives thereof; anti-migratory agents and/or derivatives thereof (e.g., caffeic acid derivatives, nilvadipine, etc.); anti-mitotic compounds and/or derivatives thereof; anti-neoplastic compounds and/or derivatives thereof; anti-oxidants and/or derivatives thereof; anti-platelet compounds and/or derivatives thereof; anti-proliferative and/or derivatives thereof; anti- thrombogenic agents and/or derivatives thereof; argatroban and/or derivatives thereof; ap-1 inhibitors and/or derivatives thereof (e.g., for tyrosine kinase, protein kinase C, myosin light chain kinase, Ca2+/calmodulin kinase II, casein kinase II, etc.); aspirin and/or derivatives thereof; azathioprine and/or derivatives thereof; $-estradiol and/or derivatives thereof; −1-anticollagenase and/or derivatives thereof; calcium channel blockers and/or derivatives thereof; calmodulin antagonists and/or derivatives thereof (e.g., H7, etc.); Captoril and/or derivatives thereof; cartilage-derived inhibitor and/or derivatives thereof; ChIMP-3 and/or derivatives thereof; cephalosporin and/or derivatives thereof (e.g., cefadroxil, cefazolin, cefaclor, etc.); chloroquine and/or derivatives thereof; chemotherapeutic compounds and/or derivatives thereof (e.g., 5-fluorouracil, vincristine, vinblastine, cisplatin, doxyrubicin, adriamycin, tamocifen, etc.); chymostatin and/or derivatives thereof; Cilazapril and/or derivatives thereof; clopidigrel and/or derivatives thereof; clotrimazole and/or derivatives thereof; colchicine and/or derivatives thereof; cortisone and/or derivatives thereof; coumadin and/or derivatives thereof; curacin-A and/or derivatives thereof; cyclosporine and/or derivatives thereof; cytochalasin and/or derivatives thereof (e.g., cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin 0, cytochalasin P, cytochalasin Q. cytochalasin R,. cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D, etc.); cytokines and/or derivatives thereof; desirudin and/or derivatives thereof; dexamethazone and/or derivatives thereof; dipyridamole and/or derivatives thereof; eminase and/or derivatives thereof; endothelin and/or derivatives thereof endothelial growth factor and/or derivatives thereof; epidermal growth factor and/or derivatives thereof; epothilone and/or derivatives thereof; estramustine and/or derivatives thereof; estrogen and/or derivatives thereof; fenoprofen and/or derivatives thereof; fluorouracil and/or derivatives thereof; flucytosine and/or derivatives thereof; forskolin and/or derivatives thereof; ganciclovir and/or derivatives thereof; glucocorticoids and/or derivatives thereof (e.g., dexamethasone, betamethasone, etc.); glycoprotein IIb/IIIa platelet membrane receptor antibody and/or derivatives thereof; GM-CSF and/or derivatives thereof; griseofulvin and/or derivatives thereof; growth factors and/or derivatives thereof (e.g., VEGF; TGF; IGF; PDGF; FGF, etc.); growth hormone and/or derivatives thereof; heparin and/or derivatives thereof; hirudin and/or derivatives thereof; hyaluronate and/or derivatives thereof; hydrocortisone and/or derivatives thereof; ibuprofen and/or derivatives thereof; immunosuppressive agents and/or derivatives thereof (e.g., adrenocorticosteroids, cyclosporine, etc.); indomethacin and/or derivatives thereof; inhibitors of the sodium/calcium antiporter and/or derivatives thereof (e.g., amiloride, etc.); inhibitors of the IP3 receptor and/or derivatives thereof; inhibitors of the sodium/hydrogen antiporter and/or derivatives thereof (e.g., amiloride and derivatives thereof, etc.); insulin and/or derivatives thereof; interferon a-2-macroglobulin and/or derivatives thereof; ketoconazole and/or derivatives thereof; lepirudin and/or derivatives thereof; Lisinopri and/or derivatives thereof; Lovastatin and/or derivatives thereof; marevan and/or derivatives thereof; mefloquine and/or derivatives thereof; metalloproteinase inhibitors and/or derivatives thereof; methotrexate and/or derivatives thereof; metronidazole and/or derivatives thereof; miconazole and/or derivatives thereof; monoclonal antibodies and/or derivatives thereof; mutamycin and/or derivatives thereof; naproxen and/or derivatives thereof; nitric oxide and/or derivatives thereof; nitroprusside and/or derivatives thereof; nucleic acid analogues and/or derivatives thereof (e.g., peptide nucleic acids, etc.); nystatin and/or derivatives thereof; oligonucleotides and/or derivatives thereof; paclitaxel and/or derivatives thereof; penicillin and/or derivatives thereof; pentamidine isethionate and/or derivatives thereof; phenindione and/or derivatives thereof; phenylbutazone and/or derivatives thereof; phosphodiesterase inhibitors and/or derivatives thereof; plasminogen activator inhibitor-1 and/or derivatives thereof; plasminogen activator inhibitor-2 and/or derivatives thereof; platelet factor 4 and/or derivatives thereof; platelet derived growth factor and/or derivatives thereof; plavix and/or derivatives thereof; POSTMI 75 and/or derivatives thereof; prednisone and/or derivatives thereof; prednisolone and/or derivatives thereof; probucol and/or derivatives thereof; progesterone and/or derivatives thereof; prostacyclin and/or derivatives thereof; prostaglandin inhibitors and/or derivatives thereof; protamine and/or derivatives thereof; protease and/or derivatives thereof; protein kinase inhibitors and/or derivatives thereof (e.g., staurosporin, etc.); quinine and/or derivatives thereof; radioactive agents and/or derivatives thereof (e.g., Cu-64, Ca-67, Cs-131, Ga-68, Zr-89, Ku-97, Tc-99m, Rh-105, Pd-103, Pd-109, In-111, 1-123, 1-125, 1-131, Re-186, Re-188, Au-198, Au-199, Pb-203, At-211, Pb-212, Bi-212, H3P3204, etc.); rapamycin and/or derivatives thereof; receptor antagonists for histamine and/or derivatives thereof; refludan and/or derivatives thereof; retinoic acids and/or derivatives thereof; revasc and/or derivatives thereof; rifamycin and/or derivatives thereof; sense or anti-sense oligonucleotides and/or derivatives thereof (e.g., DNA, RNA, plasmid DNA, plasmid RNA, etc.); seramin and/or derivatives thereof; steroids; seramin and/or derivatives thereof; serotonin and/or derivatives thereof; serotonin blockers and/or derivatives thereof; streptokinase and/or derivatives thereof; sulfasalazine and/or derivatives thereof; sulfonamides and/or derivatives thereof (e.g., sulfamethoxazole, etc.); sulphated chitin derivatives; sulphated polysaccharide peptidoglycan complex and/or derivatives thereof; THI and/or derivatives thereof (e.g., Interleukins-2, -12, and -15, gamma interferon, etc.); thioprotese inhibitors and/or derivatives thereof; taxol and/or derivatives thereof (e.g., taxotere, baccatin, 10-deacetyltaxol, 7-xylosyl- 10-deacetyltaxol, cephalomannine, 10-deacetyl-7-epitaxol, 7 epitaxol, 10-deacetylbaccatin III, 10-deacetylcephaolmannine, etc.); ticlid and/or derivatives thereof; ticlopidine and/or derivatives thereof; tick anti-coagulant peptide and/or derivatives thereof; thioprotese inhibitors and/or derivatives thereof; thyroid hormone and/or derivatives thereof; tissue inhibitor of metalloproteinase-1 and/or derivatives thereof; tissue inhibitor of metalloproteinase-2 and/or derivatives thereof; tissue plasma activators; TNF and/or derivatives thereof, tocopherol and/or derivatives thereof; toxins and/or derivatives thereof; tranilast and/or derivatives thereof; transforming growth factors alpha and beta and/or derivatives thereof; trapidil and/or derivatives thereof; triazolopyrimidine and/or derivatives thereof; vapiprost and/or derivatives thereof; vinblastine and/or derivatives thereof; vincristine and/or derivatives thereof; zidovudine and/or derivatives thereof. As can be appreciated, the agent can include one or more derivatives of the above listed compounds and/or other compounds. In one non-limiting embodiment, the agent includes, but is not limited to, trapidil, trapidil derivatives, taxol, taxol derivatives (e.g., taxotere, baccatin, 10-deacetyltaxol, 7-xylosyl-10-deacetyltaxol, cephalomannine, 10-deacetyl-7-epitaxol, 7 epitaxol, 10-deacetylbaccatin III, 10-deacetylcephaolmannine, etc.), cytochalasin, cytochalasin derivatives (e.g., cytochalasin A, cytochalasin B, cytochalasin C, cytochalasin D, cytochalasin E, cytochalasin F, cytochalasin G, cytochalasin H, cytochalasin J, cytochalasin K, cytochalasin L, cytochalasin M, cytochalasin N, cytochalasin 0, cytochalasin P, cytochalasin Q. cytochalasin R, cytochalasin S, chaetoglobosin A, chaetoglobosin B, chaetoglobosin C, chaetoglobosin D, chaetoglobosin E, chaetoglobosin F, chaetoglobosin G, chaetoglobosin J, chaetoglobosin K, deoxaphomin, proxiphomin, protophomin, zygosporin D, zygosporin E, zygosporin F, zygosporin G, aspochalasin B, aspochalasin C, aspochalasin D, etc.), paclitaxel, paclitaxel derivatives, rapamycin, rapamycin derivatives, 5-phenylmethimazole, 5-phenylmethimazole derivatives, GM-CSF (granulo-cytemacrophage colony-stimulating-factor), GM-CSF derivatives, statins or HMG-CoA reductase inhibitors forming a class of hypolipidemic agents, combinations, or analogs thereof, or combinations thereof. The type and/or amount of agent included in the device and/or coated on the device can vary. When two or more agents are included in and/or coated on the device, the amount of two or more agents can be the same or different. The type and/or amount of agent included on, in and/or in conjunction with the device are generally selected to address one or more clinical events.

Typically, the amount of agent included on, in and/or used in conjunction with the device is about 0.01-100 μg per mm 2 and/or at least about 0.01 weight percent of device; however, other amounts can be used. In one non-limiting embodiment of the disclosure, the device can be partially of fully coated and/or impregnated with one or more agents to facilitate in the success of a particular medical procedure. The amount of two of more agents on, in and/or used in conjunction with the device can be the same or different. The one or more agents can be coated on and/or impregnated in the device by a variety of mechanisms such as, but not limited to, spraying (e.g., atomizing spray techniques, etc.), flame spray coating, powder deposition, dip coating, flow coating, dip-spin coating, roll coating (direct and reverse), sonication, brushing, plasma deposition, depositing by vapor deposition, MEMS technology, and rotating mold deposition. In another and/or alternative non-limiting embodiment of the disclosure, the type and/or amount of agent included on, in and/or in conjunction with the device is generally selected for the treatment of one or more clinical events. Typically, the amount of agent included on, in and/or used in conjunction with the device is about 0.01-100 μg per mm 2 and/or at least about 0.01-100 weight percent of the device; however, other amounts can be used. The amount of two of more agents on, in and/or used in conjunction with the device can be the same or different. As such, the medical device, when it includes, contains, and/or is coated with one or more agents, can include one or more agents to address one or more medical needs. Inone non-limiting embodiment of the disclosure, the medical device can be partially or fully coated with one or more agents and/or impregnated with one or more agents to facilitate in the success of a particular medical procedure. The one or more agents can be coated on and/or impregnated in the medical device by a variety of mechanisms such as, but not limited to, spraying (e.g., atomizing spray techniques, etc.), dip coating, roll coating, sonication, brushing, plasma deposition, depositing by vapor deposition. In another and/or alternative non-limiting embodiment of the disclosure, the type and/or amount of agent included on, in and/or in conjunction with the medical device is generally selected for the treatment of one or more medical treatments. Typically, the amount of agent included on, in and/or used in conjunction with the medical device is about 0.01-100 μg per mm 2; however, other amounts can be used. The amount of two or more agents on, in and/or used in conjunction with the medical device can be the same or different.

In a further and/or alternative non-limiting aspect of the present disclosure, the one or more agents on and/or in the medical device, when used on the medical device, can be released in a controlled manner so the area in question to be treated is provided with the desired dosage of agent over a sustained period of time. As can be appreciated, controlled release of one or more agents on the medical device is not always required and/or desirable. As such, one or more of the agents on and/or in the medical device can be uncontrollably released from the medical device during and/or after insertion of the medical device in the treatment area. It can also be appreciated that one or more agents on and/or in the medical device can be controllably released from the medical device and one or more agents on and/or in the medical device can be uncontrollably released from the medical device. It can also be appreciated that one or more agents on and/or in one region of the medical device can be controllably released from the medical device and one or more agents on and/or in the medical device can be uncontrollably released from another region on the medical device. As such, the medical device can be designed such that 1) all the agent on and/or in the medical device is controllably released, 2) some of the agent on and/or in the medical device is controllably released and some of the agent on the medical device is non-controllably released, or 3) none of the agent on and/or in the medical device is controllably released. The medical device can also be designed such that the rate of release of the one or more agents from the medical device is the same or different. The medical device can also be designed such that the rate of release of the one or more agents from one or more regions on the medical device is the same or different. Non-limiting arrangements that can be used to control the release of one or more agents from the medical device include 1) at least partially coat one or more agents with one or more polymers, 2) at least partially incorporate and/or at least partially encapsulate one or more agents into and/or with one or more polymers, and/or 3) insert one or more agents in pores, passageway, cavities, etc. in the medical device and at least partially coat or cover such pores, passageway, cavities, etc. with one or more polymers. As can be appreciated, other or additional arrangements can be used to control the release of one or more agents from the medical device.

The one or more polymers used to at least partially control the release of one or more agent from the medical device can be porous or non-porous. The one or more agents can be inserted into and/or applied to one or more surface structures and/or micro-structures on the medical device, and/or be used to at least partially form one or more surface structures and/or micro-structures on the medical device. As such, the one or more agents on the medical device can be 1) coated on one or more surface regions of the medical device, 2) inserted and/or impregnated in one or more surface structures and/or micro-structures, etc. of the medical device, and/or 3) form at least a portion or be included in at least a portion of the structure of the medical device. When the one or more agents are coated on the medical device, the one or more agents can 1) be directly coated on one or more surfaces of the medical device, 2) be mixed with one or more coating polymers or other coating materials and then at least partially coated on one or more surfaces of the medical device, 3) be at least partially coated on the surface of another coating material that has been at least partially coated on the medical device, and/or 4) be at least partially encapsulated between a) a surface or region of the medical device and one or more other coating materials and/or b) two or more other coating materials.

As can be appreciated, many other coating arrangements can be additionally or alternatively used. When the one or more agents are inserted and/or impregnated in one or more internal structures, surface structures and/or micro-structures of the medical device, 1) one or more other coating materials can be applied at least partially over the one or more internal structures, surface structures and/or micro-structures of the medical device, and/or 2) one or more polymers can be combined with one or more agents. As such, the one or more agents can be 1) embedded in the structure of the medical device; 2) positioned in one or more internal structures of the medical device; 3) encapsulated between two polymer coatings; 4) encapsulated between the base structure and a polymer coating; 5) mixed in the base structure of the medical device that includes at least one polymer coating; or 6) one or more combinations of 1, 2, 3, 4 and/or 5. In addition or alternatively, the one or more coating of the one or more polymers on the medical device can include 1) one or more coatings of non-porous polymers; 2) one or more coatings of a combination of one or more porous polymers and one or more non-porous polymers; 3) one or more coating of porous polymer, or 4) one or more combinations of options 1, 2, and 3.

As can be appreciated different agents can be located in and/or between different polymer coating layers and/or on and/or the structure of the medical device. As can also be appreciated, many other and/or additional coating combinations and/or configurations can be used. The concentration of one or more agents, the type of polymer, the type and/or shape of internal structures in the medical device and/or the coating thickness of one or more agents can be used to control the release time, the release rate and/or the dosage amount of one or more agents; however, other or additional combinations can be used. As such, the agent and polymer system combination and location on the medical device can be numerous. As can also be appreciated, one or more agents can be deposited on the top surface of the medical device to provide an initial uncontrolled burst effect of the one or more agents prior to 1) the controlled release of the one or more agents through one or more layers of polymer system that include one or more non-porous polymers and/or 2) the uncontrolled release of the one or more agents through one or more layers of polymer system. The one or more agents and/or polymers can be coated on the medical device by a variety of mechanisms such as, but not limited to, spraying (e.g., atomizing spray techniques, etc.), dip coating, roll coating, sonication, brushing, plasma deposition, and/or depositing by vapor deposition.

The thickness of each polymer layer and/or layer of agent is generally at least about 0.01 μm and is generally less than about 150 μm. In one non-limiting embodiment, the thickness of a polymer layer and/or layer of agent is about 0.02-75μm, more particularly about 0.05-50 μm, and even more particularly about 1-30 μm.

When the medical device includes and/or is coated with one or more agents such that at least one of the agents is at least partially controllably released from the medical device, the need or use of body-wide therapy for extended periods of time can be reduced or eliminated. In the past, the use of body-wide therapy was used by the patient long after the patient left the hospital or other type of medical facility. This body-wide therapy could last days, weeks, months or sometimes over a year after surgery. The medical device of the present disclosure can be applied or inserted into a treatment area and 1) merely requires reduced use and/or extended use of body-wide therapy after application or insertion of the medical device, or 2) does not require use and/or extended use of body-wide therapy after application or insertion of the medical device. As can be appreciated, use and/or extended use of body-wide therapy can be used after application or insertion of the medical device at the treatment area. In one non-limiting example, no body-wide therapy is needed after the insertion of the medical device into a patient. In another and/or alternative non-limiting example, short-term use of body-wide therapy is needed or used after the insertion of the medical device into a patient. Such short-term use can be terminated after the release of the patient from the hospital or other type of medical facility, or one to two days or weeks after the release of the patient from the hospital or other type of medical facility; however, it will be appreciated that other time periods of body-wide therapy can be used. As a result of the use of the medical device of the present disclosure, the use of body-wide therapy after a medical procedure involving the insertion of a medical device into a treatment area can be significantly reduced or eliminated.

In another and/or alternative non-limiting aspect of the present disclosure, controlled release of one or more agents from the medical device, when controlled release is desired, can be accomplished by using one or more non-porous polymer layers; however, other and/or additional mechanisms can be used to controllably release the one or more agents. The one or more agents are at least partially controllably released by molecular diffusion through the one or more non-porous polymer layers. When one or more non-porous polymer layers are used, the one or more polymer layers are typically biocompatible polymers; however, this is not required. The one or more non-porous polymers can be applied to the medical device without the use of chemicals, solvents, and/or catalysts; however, this is not required. In one non-limiting example, the non-porous polymer can be at least partially applied by, but not limited to, vapor deposition and/or plasma deposition. The non-porous polymer can be selected so as to polymerize and cure merely upon condensation from the vapor phase; however, this is not required. The application of the one or more non-porous polymer layers can be accomplished without increasing the temperature above ambient temperature (e.g., 65-900F); however, this is not required. The non-porous polymer system can be mixed with one or more agents prior to being coated on the medical device and/or be coated on a medical device that previously included one or more agents; however, this is not required. The use or one or more non-porous polymer layers allow for accurate controlled release of the agent from the medical device. The controlled release of one or more agents through the non- porous polymer is at least partially controlled on a molecular level utilizing the motility of diffusion of the agent through the non-porous polymer. In one non-limiting example, the one or more non-porous polymer layers can include, but are not limited to, polyamide, parylene (e.g., parylene C, parylene N) and/or a parylene derivative.

In still another and/or alternative non-limiting aspect of the present disclosure, controlled release of one or more agents from the medical device, when controlled release is desired, can be accomplished by using one or more polymers that form a chemical bond with one or more agents. In one non-limiting example, at least one agent includes trapidil, trapidil derivative or a salt thereof, that is covalently bonded to at least one polymer such as, but not limited to, an ethylene-acrylic acid copolymer. The ethylene is the hydrophobic group and acrylic acid is the hydrophilic group. The mole ratio of the ethylene to the acrylic acid in the copolymer can be used to control the hydrophobicity of the copolymer. The degree of hydrophobicity of one or more polymers can also be used to control the release rate of one or more agents from the one or more polymers. The amount of agent that can be loaded with one or more polymers may be a function of the concentration of anionic groups and/or cationic groups in the one or more polymer. For agents that are anionic, the concentration of agent that can be loaded on the one or more polymers is generally a function of the concentration of cationic groups (e.g. amine groups and the like) in the one or more polymer and the fraction of these cationic groups that can ionically bind to the anionic form of the one or more agents. For agents that are cationic (e.g., trapidil, etc.), the concentration of agent that can be loaded on the one or more polymers is generally a function of the concentration of anionic groups (i.e., carboxylate groups, phosphate groups, sulfate groups, and/or other organic anionic groups) in the one or more polymers, and the fraction of these anionic groups that can ionically bind to the cationic form of the one or more agents. As such, the concentration of one or more agents that can be bound to the one or more polymers can be varied by controlling the amount of hydrophobic and hydrophilic monomer in the one or more polymers, by controlling the efficiency of salt formation between the agent, and/or the anionic/cationic groups in the one or more polymers.

In still another and/or alternative non-limiting aspect of the present disclosure, controlled release of one or more agents from the medical device, when controlled release is desired, can be accomplished by using one or more polymers that include one or more induced cross-links. These one or more cross-links can be used to at least partially control the rate of release of the one or more agents from the one or more polymers. The cross- linking in the one or more polymers can be instituted by a number to techniques such as, but not limited to, using catalysts, radiation, heat, and/or the like. The one or more cross-links formed in the one or more polymers can result in the one or more agents becoming partially or fully entrapped within the cross-linking, and/or form a bond with the cross-linking. As such, the partially or fully entrapped agent takes longer to release itself from the cross-linking, thereby delaying the release rate of the one or more agents from the one or more polymers. Consequently, the amount of agent, and/or the rate at which the agent is released from the medical device over time can be at least partially controlled by the amount or degree of cross-linking in the one or more polymers.

In still a further and/or alternative aspect of the present disclosure, a variety of polymers can be coated on the medical device and/or be used to form at least a portion of the medical device. The one or more polymers can be used on the medical for a variety of reasons such as, but not limited to, 1) forming a portion of the medical device, 2) improving a physical property of the medical device (e.g., improve strength, improve durability, improve biocompatibility, reduce friction, etc.), 3) forming a protective coating on one or more surface structures on the medical device, 4) at least partially forming one or more surface structures on the medical device, and/or 5) at least partially controlling a release rate of one or more agents from the medical device. As can be appreciated, the one or more polymers can have other or additional uses on the medical device. The one or more polymers can be porous, non-porous, biostable, biodegradable (i.e., dissolves, degrades, is absorbed, or any combination thereof in the body), and/or biocompatible. When the medical device is coated with one or more polymers, the polymer can include 1) one or more coatings of non-porous polymers; 2) one or more coatings of a combination of one or more porous polymers and one or more non-porous polymers; 3) one or more coatings of one or more porous polymers and one or more coatings of one or more non-porous polymers; 4) one or more coating of porous polymer, or 5) one or more combinations of options 1, 2, 3 and 4. The thickness of one or more of the polymer layers can be the same or different. When one or more layers of polymer are coated onto at least a portion of the medical device, the one or more coatings can be applied by a variety of techniques such as, but not limited to, vapor deposition and/or plasma deposition, spraying, dip-coating, roll coating, sonication, atomization, brushing and/or the like; however, other or additional coating techniques can be used. The one or more polymers that can be coated on the medical device and/or used to at least partially form the medical device can be polymers that are considered to be biodegradable, bioresorbable, or bioerodable; polymers that are considered to be biostable; and/or polymers that can be made to be biodegradable and/or bioresorbable with modification. Non-limiting examples of polymers that are considered to be biodegradable, bioresorbable, or bioerodable include, but are not limited to, aliphatic polyesters; poly(glycolic acid) and/or copolymers thereof (e.g., poly(glycolide trimethylene carbonate); poly(caprolactone glycolide)); poly(lactic acid) and/or isomers thereof (e.g., poly-L(lactic acid) and/or poly-D Lactic acid) and/or copolymers thereof (e.g. DL-PLA), with and without additives (e.g. calcium phosphate glass), and/or other copolymers (e.g. poly(caprolactone lactide), poly(lactide glycolide), poly(lactic acid ethylene glycol)); poly(ethylene glycol); poly(ethylene glycol) diacrylate; poly(lactide); polyalkylene succinate; polybutylene diglycolate; polyhydroxybutyrate (PHB); polyhydroxyvalerate (PHV); polyhydroxybutyrate/polyhydroxyvalerate copolymer (PHB/PHV); poly(hydroxybutyrate-co-valerate); polyhydroxyalkaoates (PHA); polycaprolactone; poly(caprolactone-polyethylene glycol) copolymer; poly(valerolactone); polyanhydrides; poly(orthoesters) and/or blends with polyanhydrides; poly(anhydride-co-imide); polycarbonates (aliphatic); poly(hydroxyl-esters); polydioxanone; polyanhydrides; polyanhydride esters; polycyanoacrylates; poly(alkyl 2-cyanoacrylates); poly(amino acids); poly(phosphazenes); poly(propylene fumarate); poly(propylene fumarateco-ethylene glycol); poly(fumarate anhydrides); fibrinogen; fibrin; gelatin; cellulose and/or cellulose derivatives and/or cellulosic polymers (e.g., cellulose acetate, cellulose acetate butyrate, cellulose butyrate, cellulose ethers, cellulose nitrate, cellulose propionate, cellophane); chitosan and/or chitosan derivatives (e.g., chitosan NOCC, chitosan NOOC-G); alginate; polysaccharides; starch; amylase; collagen; polycarboxylic acids; poly(ethyl ester-co-carboxylate carbonate) (and/or other tyrosine derived polycarbonates); poly(iminocarbonate); poly(BPA-iminocarbonate); poly(trimethylene carbonate); poly(iminocarbonate-amide) copolymers and/or other pseudo-poly(amino acids); poly(ethylene glycol); poly(ethylene oxide); poly(ethylene oxide)/poly(butylene terephthalate) copolymer; poly(epsilon-caprolactone-dimethyltrimethylene carbonate); poly(ester amide); poly(amino acids) and conventional synthetic polymers thereof; poly(alkylene oxalates); poly(alkylcarbonate); poly(adipic anhydride); nylon copolyamides; NO-carboxymethyl chitosan NOCC); carboxymethyl cellulose; copoly(ether-esters) (e.g., PEO/PLA dextrans); polyketals; biodegradable polyethers; biodegradable polyesters; polydihydropyrans; polydepsipeptides; polyarylates (L-tyrosine-derived) and/or free acid polyarylates; polyamides (e.g., nylon 6-6, polycaprolactam); poly(propylene fumarate-co-ethylene glycol) (e.g., fumarate anhydrides); hyaluronates; poly-p-dioxanone; polypeptides and proteins; polyphosphoester; polyphosphoester urethane; polysaccharides; pseudo-poly(amino acids); starch; terpolymer; (copolymers of glycolide, lactide, or dimethyltrimethylene carbonate); rayon; rayon triacetate; latex; and/pr copolymers, blends, and/or composites of above. Non-limiting examples of polymers that considered to be biostable include, but are not limited to, parylene; parylene c; parylene f; parylene n; parylene derivatives; maleic anyhydride polymers; phosphorylcholine; poly n-butyl methacrylate (PBMA); polyethylene-co-vinyl acetate (PEVA); PBMA/PEVA blend or copolymer; polytetrafluoroethene (Teflon®) and derivatives; poly-paraphenylene terephthalamide (Kevlar®); poly(ether ether ketone) (PEEK); poly(styrene-b-isobutylene-b-styrene) (Translute™); tetramethyldisiloxane (side chain or copolymer); polyimides polysulfides; poly(ethylene terephthalate); poly(methyl methacrylate); poly(ethylene-co-methyl methacrylate); styrene-ethylene/butylene-styrene block copolymers; ABS; SAN; acrylic polymers and/or copolymers (e.g., n-butyl-acrylate, n- butyl methacrylate, 2-ethylhexyl acrylate, lauryl-acrylate, 2-hydroxy-propyl acrylate, polyhydroxyethyl, methacrylate/methylmethacrylate copolymers); glycosaminoglycans; alkyd resins; elastin; polyether sulfones; epoxy resin; poly(oxymethylene); polyolefins; polymers of silicone; polymers of methane; polyisobutylene; ethylene-alphaolefin copolymers; polyethylene; polyacrylonitrile; fluorosilicones; poly(propylene oxide); polyvinyl aromatics (e.g. polystyrene); poly(vinyl ethers) (e.g. polyvinyl methyl ether); poly(vinyl ketones); poly(vinylidene halides) (e.g. polyvinylidene fluoride, polyvinylidene chloride); poly(vinylpyrolidone); poly(vinylpyrolidone)/vinyl acetate copolymer; polyvinylpridine prolastin or silk-elastin polymers (SELP); silicone; silicone rubber; polyurethanes (polycarbonate polyurethanes, silicone urethane polymer) (e.g., chronoflex varieties, bionate varieties); vinyl halide polymers and/or copolymers (e.g. polyvinyl chloride); polyacrylic acid; ethylene acrylic acid copolymer; ethylene vinyl acetate copolymer; polyvinyl alcohol; poly(hydroxyl alkylmethacrylate); polyvinyl esters (e.g. polyvinyl acetate); and/or copolymers, blends, and/or composites of above. Non-limiting examples of polymers that can be made to be biodegradable and/or bioresorbable with modification include, but are not limited to, hyaluronic acid (hyanluron); polycarbonates; polyorthocarbonates; copolymers of vinyl monomers; polyacetals; biodegradable polyurethanes; polyacrylamide; polyisocyanates; polyamide; and/or copolymers, blends, and/or composites of above. As can be appreciated, other and/or additional polymers and/or derivatives of one or more of the above listed polymers can be used. The one or more polymers can be coated on the medical device by a variety of mechanisms such as, but not limited to, spraying (e.g., atomizing spray techniques, etc.), dip coating, roll coating, sonication, brushing, plasma deposition, and/or depositing by vapor deposition. The thickness of each polymer layer is generally at least about 0.01 μm and is generally less than about 150 μm; however, other thicknesses can be used. In one non-limiting embodiment, the thickness of a polymer layer and/or layer of agent is about 0.02-75μm, more particularly about 0.05-50 μm, and even more particularly about 1-30 μm. As can be appreciated, other thicknesses can be used. In one non-limiting embodiment, the medical device includes and/or is coated with parylene, PLGA, POE, PGA, PLLA, PAA, PEG, chitosan and/or derivatives of one or more of these polymers. In another and/or alternative non-limiting embodiment, the medical device includes and/or is coated with a non-porous polymer that includes, but is not limited to, polyamide, parylene C, parylene N and/or a parylene derivative. In still another and/or alternative non-limiting embodiment, the medical device includes and/or is coated with poly (ethylene oxide), poly(ethylene glycol), and poly(propylene oxide), polymers of silicone, methane, tetrafluoroethylene (including TEFLON brand polymers), tetramethyldisiloxane, and the like.

In another and/or alternative non-limiting aspect of the present disclosure, the medical device, when including and/or is coated with one or more agents, can include and/or can be coated with one or more agents that are the same or different in different regions of the medical device and/or have differing amounts and/or concentrations in differing regions of the medical device. For instance, the medical device can 1) be coated with and/or include one or more biologicals on at least one portion of the medical device and at least another portion of the medical device is not coated with and/or includes agent; 2) be coated with and/or include one or more biologicals on at least one portion of the medical device that is different from one or more biologicals on at least another portion of the medical device; 3) be coated with and/or include one or more biologicals at a concentration on at least one portion of the medical device that is different from the concentration of one or more biologicals on at least another portion of the medical device; etc.

In still another and/or alternative non-limiting aspect of the present disclosure, one or more surfaces of the medical device can be treated to achieve the desired coating properties of the one or more agents and one or more polymers coated on the medical device. Such surface treatment techniques include, but are not limited to, cleaning, buffing, smoothing, etching (chemical etching, plasma etching, etc.), etc. When an etching process is used, various gasses can be used for such a surface treatment process such as, but not limited to, carbon dioxide, nitrogen, oxygen, Freon®, helium, hydrogen, etc. The plasma etching process can be used to clean the surface of the medical device, change the surface properties of the medical device so as to affect the adhesion properties, lubricity properties, etc. of the surface of the medical device. As can be appreciated, other or additional surface treatment processes can be used prior to the coating of one or more agents and/or polymers on the surface of the medical device. In one non-limiting manufacturing process, one or more portions of the medical device are cleaned and/or plasma etched; however, this is not required. Plasma etching can be used to clean the surface of the medical device, and/or to form one or more non-smooth surfaces on the medical device to facilitate in the adhesion of one or more coatings of agents and/or one or more coatings of polymer on the medical device. The gas for the plasma etching can include carbon dioxide and/or other gasses. Once one or more surface regions of the medical device have been treated, one or more coatings of polymer and/or agent can be applied to one or more regions of the medical device. For instance, 1) one or more layers of porous or non-porous polymer can be coated on an outer and/or inner surface of the medical device, 2) one or more layers of agent can be coated on an outer and/or inner surface of the medical device, or 3) one or more layers of porous or non-porous polymer that includes one or more agents can be coated on an outer and/or inner surface of the medical device. The one or more layers of agent can be applied to the medical device by a variety of techniques (e.g., dipping, rolling, brushing, spraying, particle atomization, etc.). One non-limiting coating technique is by an ultrasonic mist coating process wherein ultrasonic waves are used to break up the droplet of agent and form a mist of very fine droplets. These fine droplets have an average droplet diameter of about 0.1-3 microns. The fine droplet mist facilitates in the formation of a uniform coating thickness and can increase the coverage area on the medical device.

In still yet another and/or alternative non-limiting aspect of the present disclosure, one or more portions of the medical device can 1) include the same or different agents, 2) include the same or different amount of one or more agents, 3) include the same or different polymer coatings, 4) include the same or different coating thicknesses of one or more polymer coatings, 5) have one or more portions of the medical device controllably release and/or uncontrollably release one or more agents, and/or 6) have one or more portions of the medical device controllably release one or more agents and one or more portions of the medical device uncontrollably release one or more agents.

In yet another and/or alternative non-limiting aspect of the disclosure, the medical device can include a marker material that facilitates enabling the medical device to be properly positioned in a body passageway. The marker material is typically designed to be visible to electromagnetic waves (e.g., x-rays, microwaves, visible light, inferred waves, ultraviolet waves, etc.); sound waves (e.g., ultrasound waves, etc.); magnetic waves (e.g., MRI, etc.); and/or other types of electromagnetic waves (e.g., microwaves, visible light, inferred waves, ultraviolet waves, etc.). In one non-limiting embodiment, the marker material is visible to x-rays (i.e., radiopaque). The marker material can form all or a portion of the medical device and/or be coated on one or more portions (flaring portion and/or body portion; at ends of medical device; at or near transition of body portion and flaring section; etc.) of the medical device. The location of the marker material can be on one or multiple locations on the medical device. The size of the one or more regions that include the marker material can be the same or different. The marker material can be spaced at defined distances from one another so as to form ruler-like markings on the medical device to facilitate in the positioning of the medical device in a body passageway. The marker material can be a rigid or flexible material. The marker material can be a biostable or biodegradable material. When the marker material is a rigid material, the marker material is typically formed of a metal material (e.g., metal band, metal plating, etc.); however, other or additional materials can be used. The metal, which at least partially forms the medical device, can function as a marker material; however, this is not required. When the marker material is a flexible material, the marker material typically is formed of one or more polymers that are marker materials in-of-themselves and/or include one or more metal powders and/or metal compounds. In one non-limiting embodiment, the flexible marker material includes one or more metal powders in combinations with parylene, PLGA, POE, PGA, PLLA, PAA, PEG, chitosan and/or derivatives of one or more of these polymers. In another and/or alternative non-limiting embodiment, the flexible marker material includes one or more metals and/or metal powders of aluminum, barium, bismuth, cobalt, copper, chromium, gold, iron, stainless steel, titanium, vanadium, nickel, zirconium, niobium, lead, molybdenum, platinum, yttrium, calcium, rare earth metals, rhenium, zinc, silver, depleted radioactive elements, tantalum and/or tungsten; and/or compounds thereof. The marker material can be coated with a polymer protective material; however, this is not required. When the marker material is coated with a polymer protective material, the polymer coating can be used to 1) at least partially insulate the marker material from body fluids, 2) facilitate in retaining the marker material on the medical device, 3) at least partially shield the marker material from damage during a medical procedure and/or 4) provide a desired surface profile on the medical device. As can be appreciated, the polymer coating can have other or additional uses. The polymer protective coating can be a biostable polymer or a biodegradable polymer (e.g., degrades and/or is absorbed). The coating thickness of the protective coating polymer material, when used, is typically less than about 300 microns; however, other thickness can be used. In one non-limiting embodiment, the protective coating materials include parylene, PLGA, POE, PGA, PLLA, PAA, PEG, chitosan and/or derivatives of one or more of these polymers.

In a further and/or alternative non-limiting aspect of the present disclosure, the medical device or one or more regions of the medical device can be constructed by use of one or more MEMS techniques (e.g., 3D printing, micro-machining, laser micro-machining, laser micro-machining, micro-molding, etc.); however, other or additional manufacturing techniques can be used.

The medical device can include one or more surface structures (e.g., pore, channel, pit, rib, slot, notch, bump, teeth, needle, well, hole, groove, etc.). These structures can be at least partially formed by MEMS (e.g., micro-machining, etc.) technology and/or other types of technology.

The medical device can include one or more micro-structures (e.g., micro-needle, micro-pore, micro-cylinder, micro-cone, micro-pyramid, micro-tube, micro-parallelopiped, micro-prism, micro-hemisphere, teeth, rib, ridge, ratchet, hinge, zipper, zip-tie like structure, etc.) on the surface of the medical device. As defined herein, a micro-structure is a structure that has at least one dimension (e.g., average width, average diameter, average height, average length, average depth, etc.) that is no more than about 2mm, and typically no more than about 1 mm. As can be appreciated, the medical device, when including one or more surface structures, 1) all the surface structures can be micro-structures, 2) all the surface structures can be non-micro-structures, or 3) a portion of the surface structures can be micro- structures and a portion can be non-micro-structures. Non-limiting examples of structures that can be formed on the medical devices are illustrated in United States Patent Publication Nos. 2004/0093076 and 2004/0093077, which are incorporated herein by reference. Typically, the micro-structures, when formed, extend from or into the outer surface no more than about 400 microns, and more typically less than about 300 microns, and more typically about 15-250 microns; however, other sizes can be used. The micro-structures can be clustered together or disbursed throughout the surface of the medical device. Similar shaped and/or sized micro-structures and/or surface structures can be used, or different shaped and/or sized micro-structures can be used. When one or more surface structures and/or micro-structures are designed to extend from the surface of the medical device, the one or more surface structures and/or micro-structures can be formed in the extended position and/or be designed so as to extend from the medical device during and/or after deployment of the medical device in a treatment area. The micro-structures and/or surface structures can be designed to contain and/or be fluidly connected to a passageway, cavity, etc.; however, this is not required. The one or more surface structures and/or micro-structures can be used to engage and/or penetrate surrounding tissue or organs once the medical device has be position on and/or in a patient; however, this is not required. The one or more surface structures and/or micro-structures can be used to facilitate in forming maintaining a shape of a medical device (i.e., see devices in United States Patent Publication Nos. 2004/0093076 and 2004/0093077). The one or more surface structures and/or micro-structures can be at least partially formed by MEMS (e.g., micro-machining, laser micro-machining, micro-molding, etc.) technology; however, this is not required. In one non-limiting embodiment, the one or more surface structures and/or micro-structures can be at least partially formed of a agent and/or be formed of a polymer. One or more of the surface structures and/or micro-structures can include one or more internal passageways that can include one or more materials (e.g., agent, polymer, etc.); however, this is not required. The one or more surface structures and/or micro-structures can be formed by a variety of processes (e.g., machining, chemical modifications, chemical reactions, MEMS (e.g., micro-machining, etc.), etching, laser cutting, etc.). The one or more coatings and/or one or more surface structures and/or micro- structures of the medical device can be used for a variety of purposes such as, but not limited to, 1) increasing the bonding and/or adhesion of one or more agents, adhesives, marker materials and/or polymers to the medical device, 2) changing the appearance or surface characteristics of the medical device, and/or 3) controlling the release rate of one or more agents. The one or more micro-structures and/or surface structures can be biostable, biodegradable, etc. One or more regions of the medical device that are at least partially formed by (MEMS) techniques can be biostable, biodegradable, etc. The medical device or one or more regions of the medical device can be at least partially covered and/or filled with a protective material so to at least partially protect one or more regions of the medical device, and/or one or more micro-structures and/or surface structures on the medical device from damage.

One or more regions of the medical device, and/or one or more micro-structures and/or surface structures on the medical device can be damaged when the medical device is 1) packaged and/or stored, 2) unpackaged, 3) connected to and/or other secured and/or placed on another medical device, 4) inserted into a treatment area, 5) handled by a user, and/or 6) form a barrier between one or more micro-structures and/or surface structures and fluids in the body passageway. As can be appreciated, the medical device can be damaged in other or additional ways. The protective material can be used to protect the medical device and one or more micro-structures and/or surface structures from such damage. The protective material can include one or more polymers previously identified above. The protective material can be 1) biostable and/or biodegradable and/or 2) porous and/or non-porous.

In one non-limiting design, the polymer is at least partially biodegradable so as to at least partially expose one or more micro-structure and/or surface structure to the environment after the medical device has been at least partially inserted into a treatment area. In another and/or additional non-limiting design, the protective material includes, but is not limited to, sugar (e.g., glucose, fructose, sucrose, etc.), carbohydrate compound, salt (e.g., NaCl, etc.), parylene, PLGA, POE, PGA, PLLA, PAA, PEG, chitosan and/or derivatives of one or more of these materials; however, other and/or additional materials can be used. In still another and/or additional non-limiting design, the thickness of the protective material is generally less than about 300 microns, and typically less than about 150 microns; however, other thicknesses can be used. The protective material can be coated by one or more mechanisms previously described herein.

The present disclosure describes the use of integrated sensors and methods of manufacturing.

Example Method for Treating a Condition of the Spine

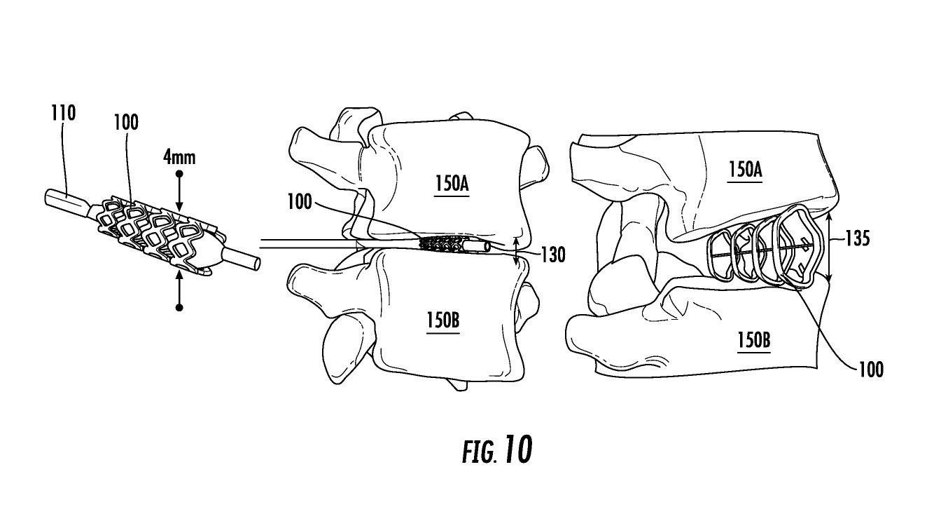

Referring now to FIG. 10, a method for treating a condition of the spine is shown. The condition of the spine can be abnormal curvature of the spine, for example. Lordotic curvature is the concave curvature of the lumbar and cervical regions of the spine (i.e., the upper back). Kyphotic curvature is the convex curvature of the thoracic and sacral regions of the spine (i.e., the lower back).

As shown in FIG. 10, a stent 100 can be inserted within a space 130 between a two vertebral bodies 150A, 150B. The stent 100 can be a catheter mounted stent. An example catheter 110 is shown in FIG. 10. The stent 100 can be expanded to move at least one of the vertebral bodies 150A, 150B. For example, the non-extended stent 100 can have a relatively small diameter (e.g., less than about 4 mm, which is provided only as an example). When the stent 100 is expanded, the diameter of the stent 100 increases. For example, the stent 100 can be a wire mesh that is crimped when not expanded. It should be understood that the stent 100 can be expanded by applying pressure internally, for example, using a balloon that can be deflated and retrieved following expansion.

The stent 100 can reset a listing height 135 between the vertebral bodies 150A, 150B. In other words, when the stent 100 is expanded within the space 130, one or more of the vertebral bodies 150A, 150B moves such that the space between the vertebral bodies 150A, 150B increases (e.g., as shown by reference number 135 in FIG. 10). Additionally, the stent 100 can reset the lordotic or kyphotic curvature of the vertebral bodies 150A, 150B as shown in FIG. 10.

Example Medical Device Having a Sensor

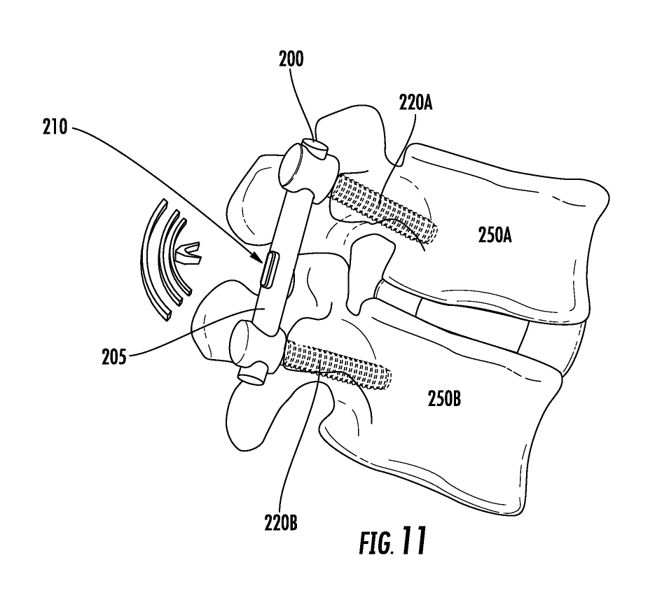

Referring now to FIG. 11, an example pedicle screw rod 200 is shown. While implementations will be described for a pedicle screw rod having a strain sensor, it will become evident to those skilled in the art that the implementations are not limited thereto, including but not limited to other medical devices having integrated sensors. Mechanical instability in a subject's spine can result from degenerative disease and/or trauma. This results in structural instability, which can cause pain. To treat such pain, a plurality of pedicle screws 220A, 220B can be inserted into respective vertebral bodies 250A, 250B of the subject's spine. The pedicle screws 220A, 220B act as firm anchor points. The pedicle screws 220A, 220B can be connected with the pedicle screw rod 200. This will eliminate movement across the unstable portion of the subject's spine and reduce or eliminate pain.

The pedicle screw rod 200 can include an elongate rod 205 and a sensor 210. The sensor 210 can be configured to measure strain in the elongate rod 205 as a function of fusion of one or more vertebral bodies 250A, 250B. Optionally, the pedicle screw rod 200 can include a plurality of sensors 210. The sensor 210 can be configured to measure compression, flexion, and/or torsion of the elongate rod 205. The sensor 210 can be configured to measure strain with micro-level precision. For example, the sensor 210 can be a strain gauge. Alternatively or additionally, the sensor 210 can include one or more piezoelectric or piezoresistive elements. Strain gauges, piezoelectric elements, and piezoresistive elements change electrical characteristics as a material deforms. Strain gauges, piezoelectric elements, and piezoresistive elements are well known in the art and are therefore not described in more detail below.

As shown in FIG. 11, the sensor 210 can be attached to or on a portion of the elongate rod 205. Alternatively or additionally, the sensor 210 can be integrated with a surface of the elongate rod 205. For example, strain gauges, piezoelectric elements, and/or piezoresistive elements can be diffused, deposited or implanted on the surface of the elongate rod 205.

In some implementations, the pedicle screw rod 200 can be used to enable real time, dynamic monitoring of conditions of the subject's spine. For example, the pedicle screw rod 200 can include measurement and/or transmission circuitry. This circuitry can be used to measure the change in electrical characteristics of the sensor 210. This circuitry can also be used to store and/or transmit the measurements, for example, to a remote computing device. This disclosure contemplates that the pedicle screw rod 200 and the remote computing device can be communicatively connected by a communication link. The communication link can be any suitable communication link. For example, a communication link can be implemented by any medium that facilitates data exchange between the network elements including, but not limited to, wired, wireless and optical links. Example communication links include, but are not limited to, a LAN, a WAN, a MAN, Ethernet, the Internet, or any other wired or wireless link such as Bluetooth, Wi-Fi, ZigBee, Wi-Max, 3G or 4G.

Example Intervertebral Spacer Having a Sensor

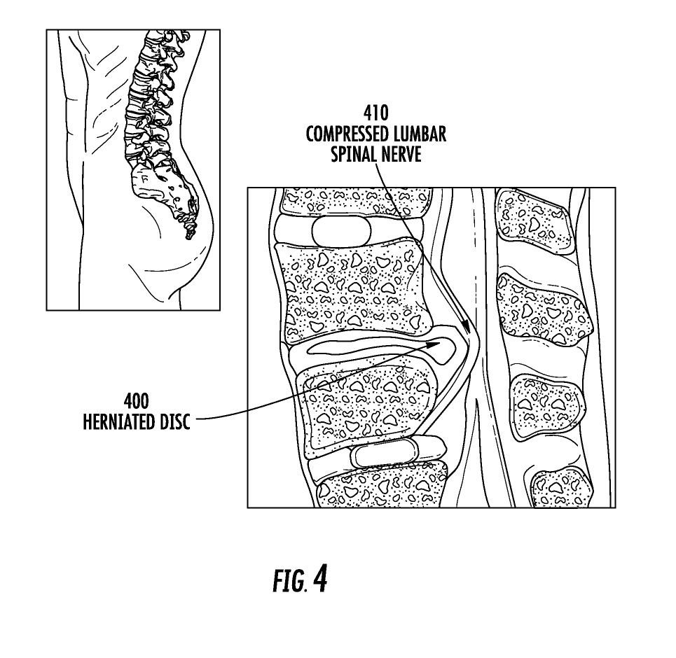

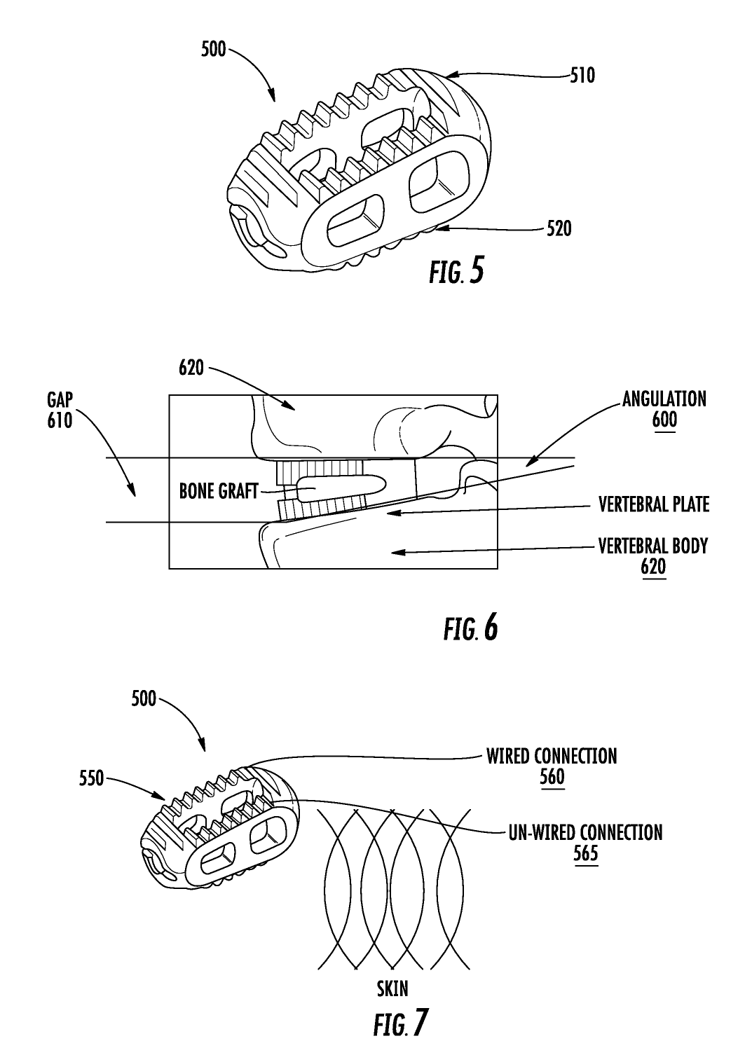

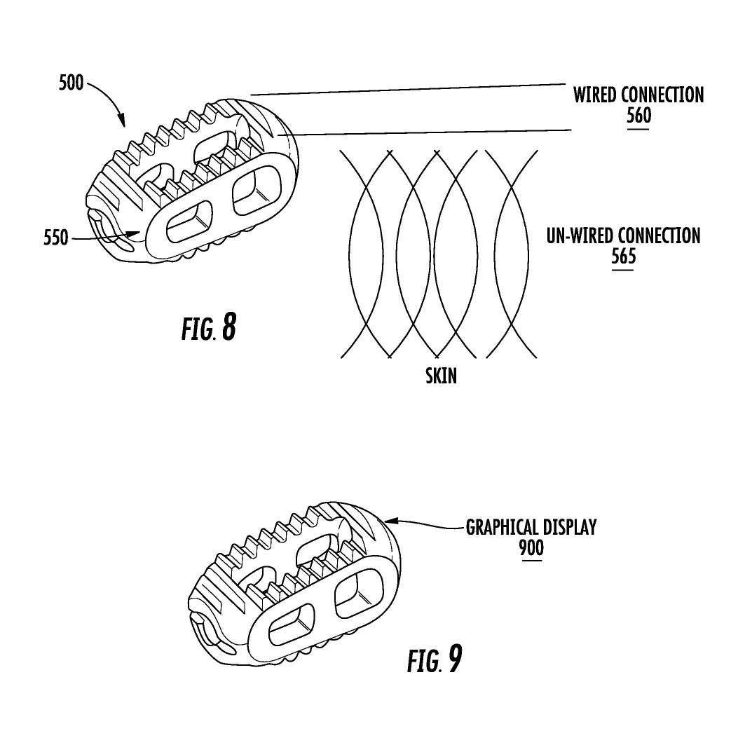

Referring now to FIGS. 5, 7, and 8, an example intervertebral spacer 500 is shown. For example, the intervertebral spacer 500 can be a spinal cage. It should be understood that the intervertebral spacer 500 described herein should not be limited to a spinal cage. As shown in FIGS. 7 and 8, the intervertebral spacer 500 includes a structure (e.g., a spinal cage) configured for insertion between vertebral bodies to reset listing height and a sensor 550 configured to measure fusion of one or more vertebral bodies as a function of strain. The sensor 550 can be configured to measure compression, flexion, and/or torsion of the intervertebral spacer 500. The sensor 550 can be configured to measure strain with micro-level precision. For example, the sensor 550 can be a strain gauge. Alternatively or additionally, the sensor 550 can include one or more piezoelectric or piezoresistive elements. Strain gauges, piezoelectric elements, and piezoresistive elements change electrical characteristics as a material deforms. Strain gauges, piezoelectric elements, and piezoresistive elements are well known in the art and are therefore not described in more detail below. Additionally, as shown in FIGS. 7 and 8, the intervertebral spacer 500 can use of a wired 560 or wireless 565 connection to the sensor 550 transmit detected changes in pressure information externally for use in evaluating the fixation, stabilization or correction or combination thereof of the clinical modality as described within this application.

Alternatively or additionally, the sensor 550 can optionally be mounted on upper face or lower face of the intervertebral spacer 500. For example, the upper face 510 and the lower face 520 of the intervertebral spacer 500 are shown in FIG. 5. Alternatively or additionally, the sensor 550 can optionally be integrated within the intervertebral spacer 500. Alternatively or additionally, the sensor 550 can optionally be printed on the upper face or lower face of the intervertebral spacer 500 or between the upper and lower faces of the intervertebral spacer 500. For example, the upper face 510 and the lower face 520 of the intervertebral spacer 500 are shown in FIG. 5.

In some implementations, the intervertebral spacer 500 can be used to enable real time, dynamic monitoring of conditions of the subject's spine. For example, the intervertebral spacer 500 can include measurement and/or transmission circuitry. This circuitry can be used to measure the change in electrical characteristics of the sensor 550. This circuitry can also be used to store and/or transmit the measurements, for example, to a remote computing device. This disclosure contemplates that the intervertebral spacer 500 and the remote computing device can be communicatively connected by a communication link. The communication link can be any suitable communication link. For example, a communication link can be implemented by any medium that facilitates data exchange between the network elements including, but not limited to, wired, wireless and optical links. Example communication links include, but are not limited to, a LAN, a WAN, a MAN, Ethernet, the Internet, or any other wired or wireless link such as Bluetooth, Wi-Fi, ZigBee, Wi-Max, 3G or 4G.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A pedicle screw rod, comprising:
an elongate rod; and
a sensor configured to measure fusion of one or more vertebral bodies as a function of strain in the elongate rod.

2. The pedicle screw rod of claim 1, wherein the sensor is attached to or on a portion of the elongate rod.

3. The pedicle screw rod of claim 1, wherein the sensor is integrated with a surface of the elongate rod.

4. The pedicle screw rod of claim 1, wherein the sensor comprises a strain gauge.

5. The pedicle screw rod of claim 1, wherein the sensor comprises a piezoelectric or piezoresistive element.

6. The pedicle screw rod of claim 1, further comprising a plurality of sensors.

7. An intervertebral spacer, comprising:
a structure configured for insertion between vertebral bodies to reset listing height; and
a sensor configured to measure fusion of one or more vertebral bodies as a function of strain.

8. The intervertebral spacer of claim 7, wherein the sensor is mounted on upper or lower face of the intervertebral spacer.

9. The intervertebral spacer of claim 7, wherein the sensor is integrated within the intervertebral spacer.

10. The intervertebral spacer of claim 7, wherein the sensor is printed on the upper or lower face of the intervertebral spacer or between the upper and lower face of the intervertebral spacer.

11. The intervertebral spacer of claim 7, wherein the sensor comprises a strain gauge.

12. The intervertebral spacer of claim 7, wherein the sensor comprises a piezoelectric or piezoresistive element.

13. The intervertebral spacer of claim 7, further comprising a plurality of sensors.

14. A method for treating a condition of the spine, comprising:
inserting a stent within a space between a two vertebral bodies; and
expanding the stent to move at least one of the vertebral bodies, wherein the expanded stent resets a listing height between the vertebral bodies and curvature of the vertebral bodies.

15. The method of claim 14, wherein the curvature is lordotic curvature.

16. The method of claim 14, wherein the curvature is kyphotic curvature.

17. The method of claim 14, wherein the stent is a catheter mounted stent.

18. The method of claim 14, wherein the stent comprises a mesh pattern.

19. A medical apparatus for use in treating disease with a sensor inherent to said apparatus for the measurement and transmission of physiological parameters.

20. A medical apparatus in the form of a spinal cage with a sensor inherent to said cage for the measurement and transmission of physiological parameters.

21. A method of constructing a medical apparatus for use in treating disease with a sensor inherent to said apparatus consisting of 3D printing of one or more materials in concert to create a single finished apparatus.

22. A method of constructing a medical apparatus for use in treating disease with a sensor inherent to said apparatus consisting of 3D printing of one or more materials in concert to create a single finished apparatus, including the use of energy to sinter metallic components for the conduction of signals and greater structural integrity.

23. A method of constructing a medical apparatus for use in treating disease with a sensor inherent to said apparatus consisting of 3D printing of one or more materials in concert to create a single finished apparatus making use of conductive polymers.
